(19) United States
(12) Patent Application Publication
(10) Pub. No.: US 2023/0087136 A1
(43) Pub. Date: Mar. 23, 2023

(54) PRE-ANALYZING AND CHARACTERIZING DATA RECORD CREATED BY WEARABLE MEDICAL SYSTEM (WMS) BEFORE REVIEW BY CLINICIAN

(71) Applicant: West Affum Holdings Corp., Grand Cayman (KY)

(72) Inventors: Jaeho Kim, Redmond, WA (US); Gregory T. Kavounas, Bellevue, WA (US)

(21) Appl. No.: 17/733,763

(22) Filed: Apr. 29, 2022

Related U.S. Application Data
(60) Provisional application No. 63/246,532, filed on Sep. 21, 2021.

(51) Int. Cl. G16H 40/67; A61N 1/37

(52) U.S. Cl. CPC G16H 40/67; A61N 1/3702; A61N 1/3706

(57) ABSTRACT

In embodiments, a pre-analyzing computer receives a data record that is created by a wearable medical system (“WMS”) which may implement a wearable cardioverter defibrillator (“WCD”). The WMS has created such data records from patient data captured when the WMS has detected that the patient was having episodes of potential interest for review by clinicians. Before this review, however, the pre-analyzing computer may parse the contents of a received data record and accordingly give it a score. The score may reflect the clinician's expected preference to review this data record before or after the others. For instance, a low score may be given to data records whose contents are likely not interpretable reliably due to noise or likely of low interest after all. The pre-analyzing computer may then perform a characterizing action with reference to the data record, for facilitating the clinician to find it by its score.

### CROSS REFERENCE TO RELATED PATENT APPLICATIONS

This patent application claims priority from U.S. provisional patent application Ser. No. 63/246,532, filed on Sep. 21, 2021.

### BACKGROUND

A wearable medical system (“WMS”) is an advanced form of a medical system. A WMS typically includes one or more wearable components that a patient can wear or carry, and possibly other components that can be portable, or stationary such as base station and/or an electric charger. The WMS may also include one or more associated software packages, such as software applications (“apps”), which can be hosted by the wearable component, and/or by a mobile device, and/or by a remote computer system that is accessible via a communications network such as the internet, and so on.

A WMS typically includes a sensor that can sense when a parameter of the patient is problematic, and cause the WMS to initiate an appropriate action. The appropriate action could be for the WMS to communicate with the patient or even with a bystander, to transmit an alert to a remotely located clinician, and to even administer treatment or therapy to the patient by itself. A WMS may actually include more than one sensor, which may sense more than one parameter of the patient. The multiple parameters may be used for determining whether or not to administer the treatment or therapy, or be suitable for detecting different problems and/or for administering respectively different treatments or therapies to the patient.

A WMS may also include the appropriate components for implementing a wearable cardioverter defibrillator (“WCD”), a pacer, and so on. Such a WMS can be for patients who have an increased risk of sudden cardiac arrest (“SCA”). In particular, when people suffer from some types of heart arrhythmias, the result may be that blood flow to various parts of the body is reduced. Some arrhythmias may result in SCA, which can lead to death very quickly, unless treated within a short time, such as 10 minutes. Some observers may have thought that SCA is the same as a heart attack, but it is not. For such patients, an external cardiac defibrillator can deliver a shock through the heart, and restore its normal rhythm. The problem is that it is hard for an external cardiac defibrillator to be brought to the patient within that short time. One solution, therefore, is for such patients to be given a WMS that implements a WCD. This solution is at least temporary and, after a while such as two months, the patient may instead receive a surgically implantable cardioverter defibrillator (“ICD”), which would then become a permanent solution.

A WMS that implements a WCD typically includes a harness, vest, belt, or other garment that the patient is to wear. The WMS system further includes additional components that are coupled to the harness, vest, or other garment. Alternately, these additional components may be adhered to the patient's skin by adhesive. These additional components include a unit that has a defibrillator, and sensing and therapy electrodes. When the patient wears this WMS, the sensing electrodes may make good electrical contact with the patient's skin and therefore can help sense the patient's Electrocardiogram (“ECG”). If the unit detects a shockable heart arrhythmia from the ECG, then the unit delivers an appropriate electric shock to the patient's body through the therapy electrodes. The shock can pass through the patient's heart and may restore its normal rhythm, thus saving their life.

All subject matter discussed in this Background section of this document is not necessarily prior art, and may not be presumed to be prior art simply because it is presented in this Background section. Plus, any reference to any prior art in this description is not, and should not be taken as, an acknowledgement or any form of suggestion that such prior art forms parts of the common general knowledge in any art in any country. Along these lines, any recognition of problems in the prior art discussed in this Background section or associated with such subject matter should not be treated as prior art, unless expressly stated to be prior art. Rather, the discussion of any subject matter in this Background section should be treated as part of the approach taken towards the particular problem by the inventors. This approach in and of itself may also be inventive.

### BRIEF SUMMARY

The present description gives instances of computer systems, storage media that may store programs, and methods, the use of which may help overcome problems and limitations of the prior art.

In embodiments, a pre-analyzing computer receives a data record that is created by a wearable medical system (“WMS”) which may implement a wearable cardioverter defibrillator (“WCD”). The WMS has created such data records from patient data captured when the WMS has detected that the patient was having episodes of potential interest for review by clinicians. Before this review, however, the pre-analyzing computer may parse the contents of a received data record and accordingly give it a score. The score may reflect the clinician's expected preference to review this data record before or after the others. For instance, a low score may be given to data records whose contents are likely not interpretable reliably due to noise or likely of low interest after all. The pre-analyzing computer may then perform a characterizing action with reference to the data record, for facilitating the clinician to find it by its score.

In embodiments, the pre-analyzing computer pre-analyzes this way and characterizes accordingly an entire batch of received data records. An advantage or benefit can be that the clinician may now review preferentially groups of data records with similar scores. In embodiments where low scores are given to data records whose contents are likely not interpretable reliably and likely of low interest after all, the clinician can avoid reviewing those, thus saving time.

As such, it will be appreciated that results of embodiments are larger than the sum of their individual parts, and have utility.

These and other features and advantages of the claimed invention will become more readily apparent in view of the embodiments described and illustrated in this specification, namely in this written specification and the associated drawings.

### BRIEF DESCRIPTION OF THE DRAWINGS

FIG. 1 is a diagram showing a patient with sample components of a Wearable Medical System (“WMS”) that implements a wearable cardioverter defibrillator (“WCD”), and a sample pre-analyzing computer system configured to pre-analyze and characterize a data record of the patient that has been created by the WMS, according to embodiments.

FIG. 2A is a diagram showing a view of the inside of a sample garment embodiment that can be a support structure of a WMS that implements a WCD, such as that of FIG. 1.

FIG. 2B is a diagram showing a view of the outside of the sample garment of FIG. 2A.

FIG. 2C is a diagram showing a front view of how the sample garment of FIGS. 2A and 2B is intended to be worn by a patient.

FIG. 2D is a diagram showing a back view of how the sample garment of FIGS. 2A and 2B is intended to be worn by a patient.

FIG. 3 is a diagram showing a partial front view of another patient wearing a sample garment embodiment of an alternate style as worn by a patient, and which can be a support structure of a WMS that implements a WCD such as that of FIG. 1.

FIG. 4 is a diagram showing sample embodiments of electronic components of a WMS that implements a WCD, and which can be used with the garment of FIG. 2A or of FIG. 3.

FIG. 5 is a diagram showing sample components of a unit of FIG. 1, which is made according to embodiments.

FIG. 6 is a diagram showing the creation of sample data records by capturing slivers of values of patient parameters according to embodiments.

FIG. 7 is a diagram showing sample scoring of a data record according to a sorting criterion, and also how the sorting criterion itself can be trained by Artificial Intelligence (AI) training techniques from known data records to which scores have been previously assigned according to embodiments.

FIG. 8 is a diagram showing a sample sorting of data records according to their given scores according to embodiments.

FIG. 9 is a diagram showing a sample scores computer file into which score entries are made that are indicative of scores given to four data records according to embodiments.

FIG. 10 is a partial screenshot of a UI, showing a portion of a sample scores computer file such as the one of FIG. 9, according to embodiments.

FIG. 11 is a diagram showing examples of four data records, and that their filenames are changed responsive to the scoring according to embodiments.

FIG. 12 shows a table with sample filenames of sample data records, their sample given scores, a sample way as to how their filenames are changed, and how they might afterwards appear sorted in a folder thanks to their changed file names, all according to embodiments.

FIG. 13 is a diagram showing four sample data records, and how scoring notes are added to their contents according to embodiments.

FIG. 14 is a diagram showing examples of adding scoring notes to the contents of a data record as in FIG. 13, plus adding a score determination record, all according to embodiments.

FIG. 15 is a diagram showing how sample received data records can be moved to different folders according to their given scores, according to embodiments.

FIG. 16 is a diagram showing how only sample received data records that have been given a score of interest are copied to a specific folder for review, according to embodiments.

FIG. 17 is a flowchart for illustrating sample methods according to embodiments.

### DETAILED DESCRIPTION

As has been mentioned, the present description is about computer systems, storage media that may store programs, and methods. Embodiments are now described in more detail.

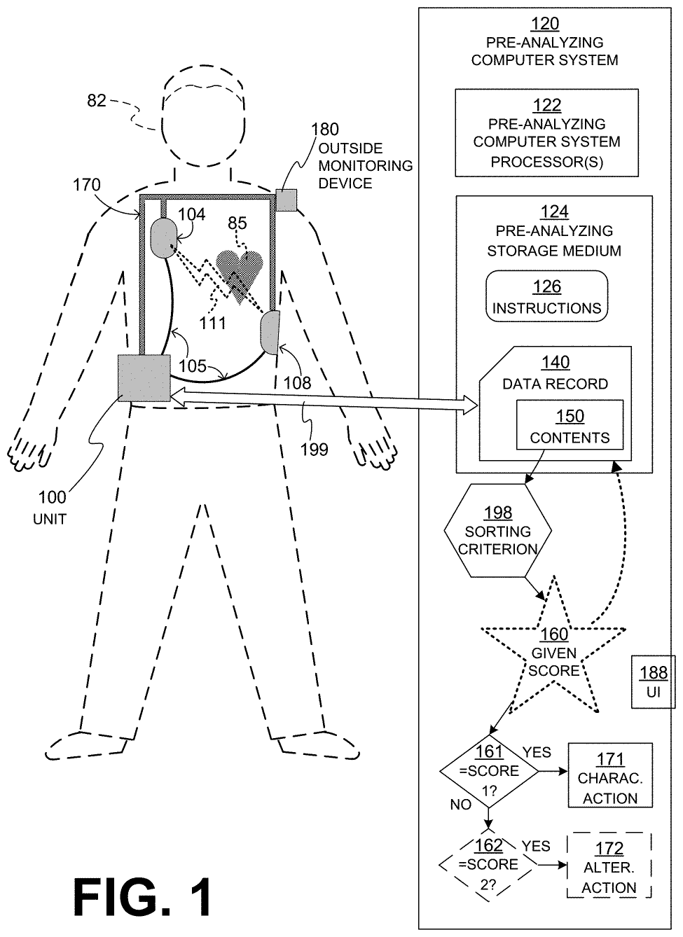

A sample pre-analyzing computer system 120 for pre-analyzing and characterizing a data record 140 is shown in FIG. 1. The data record 140 is of at least one parameter of an ambulatory patient 82. FIG. 1 also shows a components of Wearable Medical System (“WMS”) worn by the patient 82, and which created the data record 140, as described in more detailed later in this document.

The pre-analyzing computer system 120 can be a special-purpose computer associated with the WMS worn by the patient 82. For instance, the pre-analyzing computer system 120 can be a custom device that is provided together with the WMS; it can be portable or combined with a base station for the WMS, with a charger for a battery of the WMS, and so on. Or, the pre-analyzing computer system 120 can be a general-purpose computer that is running a suitable software application, and so on. The pre-analyzing computer system 120 may receive the data record 140 from the WMS via a connection 199. The connection 199 can be wired, wireless, or have elements of both. In embodiments, the pre-analyzing computer system 120 is coupled with a component of the WMS to exchange data with it. The received data record 140 can be a standalone computer file. Sometimes such a file is called an episode review file.

The pre-analyzing computer system 120 includes one or more pre-analyzing computer system processors 122. These processor(s) 122 can be distinct from the WMS processor 530 of the WMS that is described later in this document. In fact, the processor(s) 122 might not be controlled by the WMS processor 530.

The pre-analyzing computer system 120 also includes a non-transitory computer-readable pre-analyzing storage medium 124. The pre-analyzing storage medium 124 has stored thereon instructions 126. When the instructions 126 are executed by the pre-analyzing computer system processor(s) 122, they result in operations of embodiments. The data record 140 can also be stored on the pre-analyzing storage medium 124.

Additional elements are shown in FIG. 1 as part of the pre-analyzing computer system 120, and are described later in this document. Of those, it will be understood that operations may be performed by the pre-analyzing computer system processor(s) 122, and data can be stored on the pre-analyzing storage medium 124.

The pre-analyzing computer system 120 may also have a user interface (UI) 188. This could be for a user, such as a clinician, to prompt for receiving the data record 140, perhaps for managing the pre-analyzing of the data records before reviewing them, for training scoring criteria as described later in this document, for navigating among the data records after they have been characterized, for reviewing the characterized data records, and so on.

Embodiments of the wearable medical system (“WMS”) are now described in more detail. It will be understood that embodiments of the WMS may or may not implement a wearable cardioverter defibrillator (“WCD”). For instance, in embodiments where a WCD is not implemented, an arrhythmia criterion may be used instead of, or together with the alert criterion. Embodiments of the WMS that further implement a WCD may further protect an ambulatory patient by electrically restarting their heart if needed. While these latter embodiments are described in detail, such is only for explanation, and are not limiting for also covering embodiments where the WMS does not implement a WCD.

A WMS according to embodiments may have a number of components. These components can be provided separately as modules that can be interconnected, or can be combined with other components, and so on. Examples are now described.

FIG. 1 depicts a patient 82. The patient 82 may also be referred to as the person 82 and/or wearer 82, since the patient 82 is wearing components of the WMS. The patient 82 is ambulatory, which means that, while wearing the wearable component(s) of the WMS, the patient 82 can walk around, be in a vehicle, and so on. In other words, the patient 82 is not necessarily bed-ridden. While the patient 82 may be considered to be also a “user” of the WMS, this definition is not exclusive to the patient 82. For instance, a user of the WMS may also be a clinician such as a doctor, nurse, emergency medical technician (EMT), or other similarly tasked and/or empowered individual or group of individuals. In some cases, a user may even be a bystander. The particular context of these and other related terms within this description should be interpreted accordingly.

A WMS that implements a WCD according to embodiments can be configured to defibrillate the patient who is wearing the designated component(s) of the WMS. Defibrillating can be by the WMS delivering an electrical charge to the patient's body in the form of an electric shock. The electric shock can be delivered in one or more pulses.

In particular, FIG. 1 also depicts components of a WMS that implements a WCD and is made according to embodiments. One such component is a support structure 170 that is wearable by the ambulatory patient 82. Accordingly, the support structure 170 can be configured to be worn by the ambulatory patient 82 for at least several hours per day, and also during the night. That, for at least several days, and may be even a few months. It will be understood that the support structure 170 is shown only generically in FIG. 1, and in fact partly conceptually. FIG. 1 is provided merely to illustrate concepts about the support structure 170, and is not to be construed as limiting how the support structure 170 is implemented, or how it is worn.

The support structure 170 can be implemented in many different ways. For example, it can be implemented in a single component or a combination of multiple components. In embodiments, the support structure 170 could include a vest, a half-vest, a garment, etc. In such embodiments such items can be worn similarly to analogous articles of clothing. In embodiments, the support structure 170 could include a harness, one or more belts or straps, etc. In such embodiments, such items can be worn by the patient around the torso, hips, over the shoulder, etc. In embodiments, the support structure 170 can include a container or housing, which can even be waterproof. In such embodiments, the support structure can be worn by being attached to the patient's body by adhesive material, for example as shown and described in U.S. Pat. No. 8,024,037. The support structure 170 can even be implemented as described for the support structure of US Pat. App. No. US2017/0056682, which is incorporated herein by reference. Of course, in such embodiments, the person skilled in the art will recognize that additional components of the WMS can be in the housing of a support structure instead of being attached externally to the support structure, for example as described in the US2017/0056682 document. There can be other examples.

The embodiments of FIG. 1 include a sample unit 100. In embodiments, the unit 100 is sometimes called a main electronics module. In embodiments, the unit 100 implements an external defibrillator. In embodiments, the unit 100 implements an external pacer instead of, or in addition to, an external defibrillator. In embodiments that include a pacer, the WMS may detect when the patient's heart rhythm slows down or when the patient has asystole, and the pacer may pace to increase the heart rate. In such embodiments, the WMS may pace the patient first, and hopefully not have to resort to the full intervention of defibrillation. Of course, if the patient does not respond to the pacing and their heart rhythm deteriorates further, the WMS may then later cause one or more defibrillation shocks to be delivered.

The embodiments of FIG. 1 also include sample therapy electrodes 104, 108, which are electrically coupled to unit 100 via electrode leads 105. The therapy electrodes 104, 108 are also called defibrillation electrodes or just electrodes. The therapy electrodes 104, 108 can be configured to be worn by the patient 82 in a number of ways. For instance, the unit 100 and the therapy electrodes 104, 108 can be coupled to the support structure 170, directly or indirectly. In other words, the support structure 170 can be configured to be worn by the ambulatory patient 82 so as to maintain at least one of the therapy electrodes 104, 108 on the body of the ambulatory patient 82, while the patient 82 is moving around, etc. The therapy electrodes 104, 108 can be thus maintained on the body by being attached to the skin of the patient 82, simply pressed against the skin directly or through garments, etc. In some embodiments the therapy electrodes 104, 108 are not necessarily pressed against the skin, but become biased that way upon sensing a condition that could merit intervention by the WMS. In addition, many of the components of the unit 100 can be considered coupled to the support structure 170 directly, or indirectly via at least one of the therapy electrodes 104, 108.

When the therapy electrodes 104, 108 make good electrical contact with the body of the patient 82, the unit 100 can administer, via the therapy electrodes 104, 108, a brief, strong electric pulse 111 through the body. The pulse 111 is also known as defibrillation pulse, shock, defibrillation shock, therapy, electrotherapy, therapy shock, etc. The pulse 111 is intended to go through and restart the heart 85, in an effort to save the life of the patient 82. The defibrillation pulse 111 can have an energy suitable for its purpose, such as at least 100 Joule (“J”), 200 J, 300 J, and so on. For pacer embodiments, the pulse 111 could alternately be depicting a pacing pulse. At least some of the stored electrical charge can be caused to be discharged via at least two of the therapy electrodes 104, 108 through the ambulatory patient 82, so as to deliver to the ambulatory patient 82 a pacing sequence of pacing pulses. The pacing pulses may be periodic, and thus define a pacing period and the pacing rate. There is no requirement, however, that the pacing pulses be exactly periodic. A pacing pulse can have an energy suitable for its purpose, such as at most 10 J, 5 J, usually about 2 J, and so on. The pacer therefore is delivering current to the heart to start a heartbeat. In either case, the pulse 111 has a waveform suitable for this purpose.

A prior art defibrillator typically decides whether to defibrillate or not based on an ECG signal of the patient. However, the unit 100 may initiate defibrillation, or hold-off defibrillation, based on a variety of inputs, with the ECG signal merely being one of these inputs.

A WMS that implements a WCD according to embodiments can collect data about one or more parameters of the patient 82. For collecting such data, the WMS may optionally include at least an outside monitoring device 180. The device 180 is called an “outside” device because it could be provided as a standalone device, for example not within the housing of the unit 100. The device 180 can be configured to sense or monitor at least one local parameter. A local parameter can be a parameter of the patient 82, or a parameter of the WMS, or a parameter of the environment, as described later in this document.

For some of these parameters, the device 180 may include one or more sensors or transducers. Each one of such sensors can be configured to sense a parameter of the patient 82, or of the environment, and to render an input responsive to the sensed parameter. In some embodiments the input is quantitative, such as values of a sensed parameter; in other embodiments the input is qualitative, such as informing whether or not a threshold is crossed, and so on. Such inputs about the patient 82 are also called physiological inputs and patient inputs. In embodiments, a sensor can be construed more broadly, as encompassing more than one individual sensors.

Optionally, the device 180 is physically coupled to the support structure 170. In addition, the device 180 may be communicatively coupled with other components that are coupled to the support structure 170, such as with the unit 170. Such communication can be implemented by the device 180 itself having a communication module, as will be deemed applicable by a person skilled in the art in view of this description.

A WMS that implements a WCD according to embodiments preferably includes sensing electrodes, which can sense an ECG of the patient. In embodiments, the device 180 stands for such sensing electrodes. In those embodiments, the sensed parameter of the patient 82 is the ECG of the patient, the rendered input can be time values of a waveform of the ECG signal, and so on.

In embodiments, one or more of the components of the shown WMS may be customized for the patient 82. This customization may include a number of aspects. For instance, the support structure 170 can be fitted to the body of the patient 82. For another instance, baseline physiological parameters of the patient 82 can be measured for various scenarios, such as when the patient is lying down (various orientations), sitting, standing, walking, running, and so on. These baseline physiological parameters can be the heart rate of the patient 82, motion detector outputs, one for each scenario, etc. The measured values of such baseline physiological parameters can be used to customize the WMS, in order to make its diagnoses more accurate, since patients' bodies differ from one another. Of course, such parameter values can be stored in a memory of the WMS, and so on. Moreover, a programming interface can be made according to embodiments, which receives such measured values of baseline physiological parameters. Such a programming interface may input automatically these in the WMS, along with other data.

The support structure 170 is configured to be worn by the ambulatory patient 82 so as to maintain the therapy electrodes 104, 108 on a body of the patient 82. As mentioned before, the support structure 170 can be advantageously implemented by clothing or one or more garments. Such clothing or garments do not have the function of covering a person's body as a regular clothing or garments do, but the terms “clothing” and “garment” are used in this art for certain components of the WMS intended to be worn on the human body in the same way as clothing and garments are. In fact, such clothing and garments of a WMS can be of different sizes for different patients, and even be custom-fitted around the human body. Examples are now described. And, regular clothing can often be worn over portions or all of the support structure 170. Examples of the support structure 170 are now described.

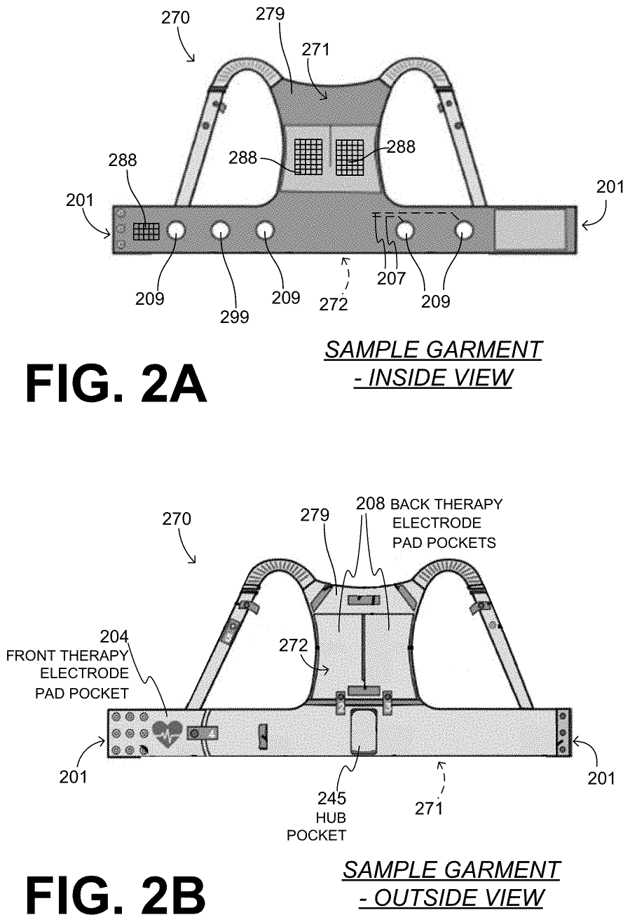

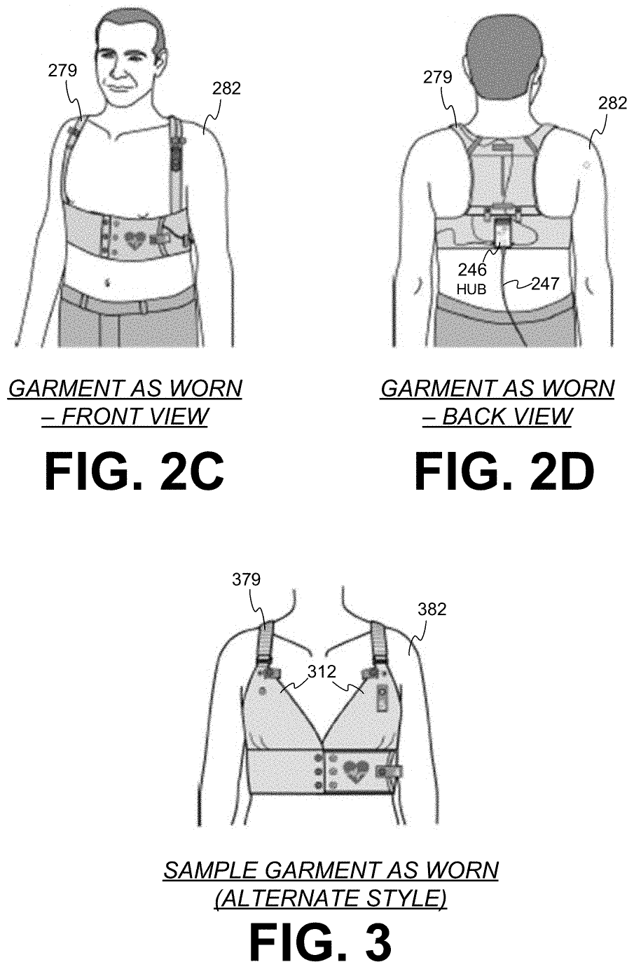

FIG. 2A shows a support structure 270 of a WMS that implements a WCD, such as the support structure 170 of FIG. 1. The support structure 270 is implemented by a vest-like wearable garment 279 that is shown flat, as if placed on a table. The inside side 271 of the garment 279 is seen as one looks at the diagram from the top, and it is the side contacting the body of the wearer when the garment 279 is worn. The outside side 272 of the garment 279 is opposite the inside side 271. To be worn, tips 201 can be brought together while surrounding the torso, and affixed to each other, either at their edges or partly overlapping. Appropriate mechanisms can hold together the tips 201, such as hooks and loops, Velcro® material, and so on.

The garment 279 can be made of suitable combinations of materials, such as fabric, linen, plastic, and so on. In places, the garment 279 can have two adjacent surfaces for defining between them pockets for the pads of the electrodes, for enclosing the leads or wires of the electrodes, and so on. Moreover, in FIG. 2A one can see meshes 288 which are the interior side of pockets accessible from the outside. The meshes can be made from flexible material such as loose netting, and so on.

ECG signals in a WMS that implements a WCD may sometimes include too much electrical noise for analyzing the ECG signal. To ameliorate the problem, multiple ECG sensing electrodes are provided in embodiments. These multiple ECG sensing electrodes define different vectors for sensing ECG signals along different ECG channels. These different ECG channels therefore present alternative options for analyzing the patient's ECG signal. The patient impedance along each ECG channel may also be sensed, and thus be part of the patient input.

In the example of FIG. 2A, multiple ECG sensing electrodes 209 are provided, which can be seen protruding from the inside surface of the garment 279. These ECG sensing electrodes 209 can be affixed to the inside surface of the garment 279, while their leads or wires 207 can be located mostly or completely within the garment 279. These ECG sensing electrodes 209 are intended to contact the skin of the person when the garment 279 is worn, and can be made from suitable material for good electrical contact. Such a material can be a metal, such as silver. An additional ECG-sensing electrode 299 may play the role of a Right Leg Drive (“RLD”) in the ECG analysis. It will be understood that “RLD” is a name for a specific ECG lead, and embodiments do not require that the electrode 299 be actually placed on the patient's right leg.

FIG. 2B shows the outside side 272 of the garment 279. One can appreciate that pockets are included that are accessible from the outside, such as a hub pocket 245. In addition a pocket 204 is provided for a front therapy electrode pad, plus two pockets 208 are provided for two back therapy electrode pads. The pads of the therapy electrodes can be placed in the pockets 204, 208, and contact the skin of the patient through the respective meshes 288 that were seen in FIG. 2A. The electrical contact can be facilitated by conductive fluid that can be deployed in the area, when the time comes for a shock.

FIG. 2C is a diagram showing a front view of how the garment 279 would be worn by a patient 282. It will be appreciated that the previously described ECG sensing electrodes 209, 299 of FIG. 2A are maintained against the body of the patient 282 from the inside side of the garment 279, and thus are not visible in FIG. 2C.

FIG. 2D is a diagram showing the back view of FIG. 2C. A hub 246 has been placed in the hub pocket 245 that is shown in FIG. 2B. A cable 247 emerges from the hub 246, which can be coupled with a unit for the system, as described later in this document.

FIGS. 2A-2D do not show any physical support for a unit such as the unit 100 of FIG. 1. In these embodiments, such a unit may be carried in a purse, on a belt, by a strap over the shoulder, or additionally by further adapting the garment 279, and so on.

FIG. 3 is a diagram showing a partial front view of another patient 382 wearing another garment 379. The garment 379 is of an alternate style than the garment 279, in that it further includes breast support receptacles 312, as was described for instance in U.S. Pat. No. 10,926,080. This style of garment may be more comfortable if the patient 382 is a woman.

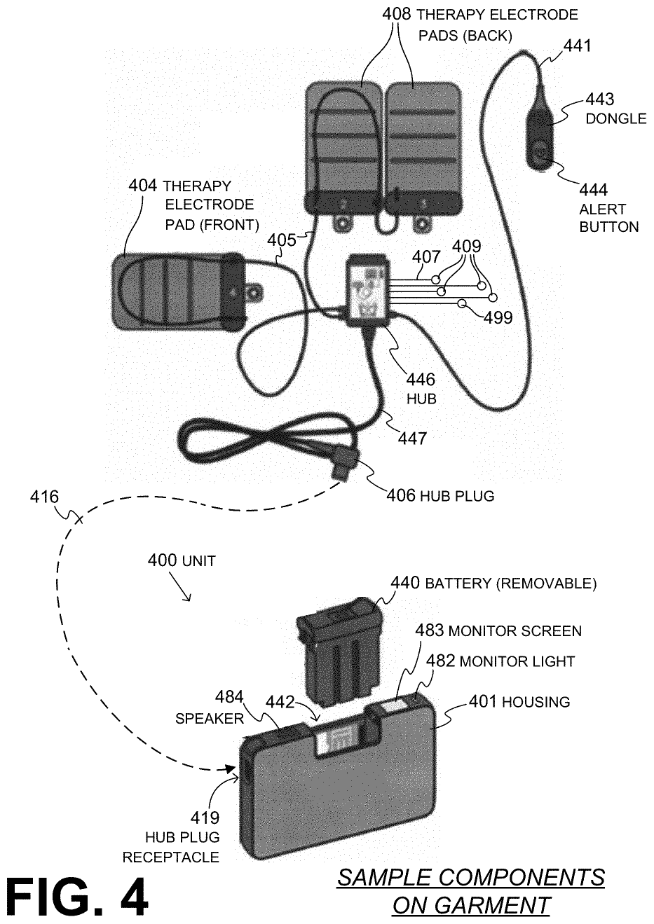

FIG. 4 shows sample electronic components that can be used with the garments 279, 379. The components of FIG. 4 include a unit 400, shown at the lower portion of FIG. 4. The unit 400 includes a housing 401, and a hub plug receptacle 419 at the housing 401.

The unit 400 includes a battery opening 442 at the housing 401. The battery opening 442 is configured to receive a removable battery 440. A system according to embodiments can have two identical such batteries 440, one plugged into the housing 401 while another one (not shown) is being charged by a charger (not shown). The batteries can then be interchanged when needed.

The unit 400 also includes devices for implementing a user interface. In this example, these devices include a monitor light 482, a monitor screen 483 and a speaker 484. Additional devices may include a vibrating mechanism, and so on.

The unit 400 can implement many of the functions of the unit 100 of FIG. 1. In the embodiment of FIG. 4, however, some of the functions of the unit 100 are implemented instead by a separate hub 446, which can be connected to the unit 400. The hub 446 is smaller and lighter than the unit 400, and can accommodate multiple electrical connections to other components of FIG. 4. A cable 447, similar to the cable 247 of FIG. 2, emerges from the hub 446 and terminates in a hub plug 406. The hub plug 406 can be plugged into the hub plug receptacle 419 of the unit 400 according to an arrow 416.

ECG sensing electrodes 409, 499, plus their wires or leads 407 are further shown conceptually in FIG. 4 for completeness. The wires or leads 407 that can be configured to be coupled to the hub 446.

The components of FIG. 4 also include the therapy electrode pads 404, 408. The therapy electrode pad 404 can be inserted into the pocket 204 of FIG. 2B, while the therapy electrode pads 408 can be inserted into the pockets 208 of FIG. 2B. The shock is generated between the therapy electrode pad 404 and the therapy electrode pads 408 taken together. Indeed, the therapy electrode pads 408 are electrically connected to each other. The therapy electrode pads 404, 408, have leads 405, which can be configured to be coupled to the hub 446.

The components of FIG. 4 further include a dongle 443 with an alert button 444. The dongle 443 can be configured to be coupled to the hub 446 via a cable 441. The alert button 444 can be used by the patient to give emergency input to the WMS. For instance, the alert button 444 can be used by the patient to notify the system that the patient is actually alive and an imminent shock is not actually needed, which may otherwise happen in the event of a false positive detection of a shockable heart rhythm of the patient.

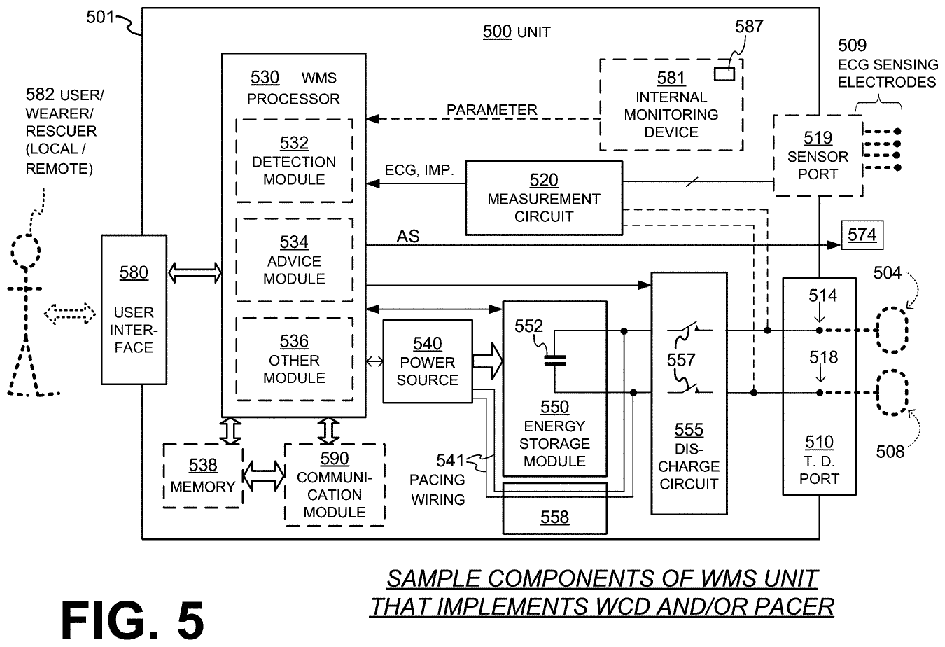

FIG. 5 shows a sample unit 500, which could be the unit 100 of FIG. 1. The unit 500 implements an external defibrillator and/or a pacer. The sample unit 500 thus combines the functions of the unit 400 and of the hub 446 of FIG. 4. The components shown in FIG. 5 can be provided in a housing 501, which may also be referred to as casing 501.

The unit 500 may include a user interface (UI) 580 for a user 582. User 582 can be the patient 82, also known as patient 582, also known as the wearer 582. Or, the user 582 can be a local rescuer at the scene, such as a bystander who might offer assistance, or a trained person. Or, the user 582 might be a remotely located trained caregiver in communication with the WMS, such as a clinician.

The user interface 580 can be made in a number of ways. The user interface 580 may include output devices, which can be visual, audible or tactile, for communicating to a user by outputting images, sounds or vibrations. Images, sounds, vibrations, and anything that can be perceived by user 582 can also be called human-perceptible indications. As such, an output device according to embodiments can be configured to output a human-perceptible indication (HPI). Such HPIs can be used to alert the patient, sound alarms that may be intended also for bystanders, and so on. There are many instances of output devices. For example, an output device can be a light that can be turned on and off, a screen to display what is sensed, detected and/or measured, and provide visual feedback to the local rescuer 582 for their resuscitation attempts, and so on. Another output device can be a speaker, which can be configured to issue voice prompts, alerts, beeps, loud alarm sounds and/or words, and so on. These can also be for bystanders, when defibrillating or just pacing, and so on. Examples of output devices were the monitor light 482, the monitor screen 483 and the speaker 484 of the unit 400 seen in FIG. 4.

The user interface 580 may further include input devices for receiving inputs from users. Such users can be the patient 82, 582, perhaps a local trained caregiver or a bystander, and so on. Such input devices may include various controls, such as pushbuttons, keyboards, touchscreens, one or more microphones, and so on. An input device can be a cancel switch, which is sometimes called an “I am alive” switch or “live man” switch. In some embodiments, actuating the cancel switch can prevent the impending delivery of a shock, or of pacing pulses. In particular, in some embodiments a speaker of the WMS is configured to output a warning prompt prior to an impending or planned defibrillation shock or a pacing sequence of pacing pulses being caused to be delivered, and the cancel switch is configured to be actuated by the ambulatory patient 82 in response to the warning prompt being output. In such embodiments, the impending or planned defibrillation shock or pacing sequence of the pacing pulses is not caused to be delivered. An example of a cancel switch was the alert button 444 seen in FIG. 4.

The unit 500 may include an internal monitoring device 581. The device 581 is called an “internal” device because it is incorporated within the housing 501. The monitoring device 581 can sense or monitor patient parameters such as patient physiological parameters, system parameters and/or environmental parameters, all of which can be called patient data. In other words, the internal monitoring device 581 can be complementary of, or an alternative to, the outside monitoring device 180 of FIG. 1. Allocating which of the parameters are to be monitored by which of the monitoring devices 180, 581 can be done according to design considerations. The device 581 may include one or more sensors, as also described elsewhere in this document.

Patient parameters may include patient physiological parameters. Patient physiological parameters may include, for example and without limitation, those physiological parameters that can be of any help in detecting by the WMS whether or not the patient is in need of a shock or other intervention or assistance. Patient physiological parameters may also optionally include the patient's medical history, event history and so on. Examples of such parameters include the patient's ECG, blood oxygen level, blood flow, blood pressure, blood perfusion, pulsatile change in light transmission or reflection properties of perfused tissue, heart sounds, heart wall motion, breathing sounds and pulse. Accordingly, the monitoring devices 180, 581 may include one or more sensors or transducers configured to acquire patient physiological signals. Examples of such sensors and transducers include the above-described electrodes to detect the ECG, a perfusion sensor, a pulse oximeter, a device for detecting blood flow (e.g. a Doppler device), a sensor for detecting blood pressure (e.g. a cuff), an optical sensor, illumination detectors and sensors perhaps working together with light sources for detecting color change in tissue, a motion sensor, a device that can detect heart wall movement, a sound sensor, a device with a microphone, an SpO2 sensor, and so on. In view of this disclosure, it will be appreciated that such sensors can help detect the patient's pulse, and can therefore also be called pulse detection sensors, pulse sensors, and pulse rate sensors. In addition, a person skilled in the art may implement other ways of performing pulse detection.

In some embodiments, the local parameter reflects a trend that can be detected in a monitored physiological parameter of the patient 82, 582. Such a trend can be detected by comparing values of parameters at different times over short and long terms. Parameters whose detected trends can particularly help a cardiac rehabilitation program include: a) cardiac function (e.g. ejection fraction, stroke volume, cardiac output, etc.); b) heart rate variability at rest or during exercise; c) heart rate profile during exercise and measurement of activity vigor, such as from the profile of an accelerometer signal and informed from adaptive rate pacemaker technology; d) heart rate trending; e) perfusion, such as from SpO2, CO2, or other parameters such as those mentioned above, f) respiratory function, respiratory rate, etc.; g) motion, level of activity; and so on. Once a trend is detected, it can be stored and/or reported via a communication link, along perhaps with a warning if warranted. From the report, a physician monitoring the progress of the patient 82, 582 will know about a condition that is either not improving or deteriorating.

Patient state parameters include recorded aspects of the patient 582, such as motion, posture, whether they have spoken recently plus may be also what they said, and so on, plus optionally the history of these parameters. Or, one of these monitoring devices could include a location sensor such as a Global Positioning System (GPS) location sensor. Such a sensor can detect the location, plus a speed of the patient can be detected as a rate of change of location over time. Many motion detectors output a motion signal that is indicative of the motion of the detector, and thus of the patient's body. Patient state parameters can be very helpful in narrowing down the determination of whether SCA is indeed taking place.

A WMS made according to embodiments may thus include a motion detector. In embodiments, a motion detector can be implemented within the outside monitoring device 180 or within the internal monitoring device 581. A motion detector of a WMS according to embodiments can be configured to detect a motion event. A motion event can be defined as is convenient, for example a change in posture or motion from a baseline posture or motion, etc. In such cases, a sensed patient parameter is motion. Such a motion detector can be made in many ways as is known in the art, for example by using an accelerometer and so on. In this example, a motion detector 587 is implemented within the monitoring device 581.

System parameters of a WMS can include system identification, battery status, system date and time, reports of self-testing, records of data entered, records of episodes and intervention, and so on. In response to the detected motion event, the motion detector may render or generate, from the detected motion event or motion, a motion detection input that can be received by a subsequent device or functionality.

Environmental parameters can include ambient temperature and pressure. Moreover, a humidity sensor may provide information as to whether or not it is likely raining. Presumed patient location could also be considered an environmental parameter. The patient location could be presumed, if the monitoring device 180 or 581 includes a GPS location sensor as per the above, and if it is presumed or sensed that the patient is wearing the WMS.

The unit 500 includes a therapy delivery port 510 and a sensor port 519 in the housing 501. In contrast, in FIG. 4 these ports are located at the hub 446.

In FIG. 5, the therapy delivery port 510 can be a socket in the housing 501, or other equivalent structure. The therapy delivery port 510 includes electrical nodes 514, 518. Therapy electrodes 504, 508 are shown, which can be as the therapy electrodes 104, 108. Leads of the therapy electrodes 504, 508, such as the leads 105 of FIG. 1, can be plugged into the therapy delivery port 510, so as to make electrical contact with the nodes 514, 518, respectively. It is also possible that the therapy electrodes 504, 508 are connected continuously to the therapy delivery port 510, instead. Either way, the therapy delivery port 510 can be used for guiding, via electrodes, to the wearer at least some of the electrical charge that has been stored in an energy storage module 550 that is described more fully later in this document. When thus guided, the electric charge will cause the shock 111 to be delivered.

The sensor port 519 is also in the housing 501, and is also sometimes known as an ECG port. The sensor port 519 can be adapted for plugging in the leads of ECG sensing electrodes 509. The ECG sensing electrodes 509 can be as the ECG sensing electrodes 209. The ECG sensing electrodes 509 in this example are distinct from the therapy electrodes 504, 508. It is also possible that the sensing electrodes 509 can be connected continuously to the sensor port 519, instead. The electrodes 509 can be types of transducers that can help sense an ECG signal of the patient, e.g. a 12-lead signal, or a signal from a different number of leads, especially if they make good electrical contact with the body of the patient and in particular with the skin of the patient. As with the therapy electrodes 504, 508, the support structure can be configured to be worn by the patient 582 so as to maintain the sensing electrodes 509 on a body of the patient 582. For example, the sensing electrodes 509 can be attached to the inside of the support structure 170 for making good electrical contact with the patient, similarly with the therapy electrodes 504, 508. The ECG sensing electrodes 509 are themselves examples of sensors configured to sense a parameter of the patient, the parameter including an Electrocardiogram (ECG) signal of the patient.

Optionally a WMS according to embodiments also includes a fluid that it can deploy automatically between the electrodes and the patient's skin. The fluid can be conductive, such as by including an electrolyte, for establishing a better electrical contact between the electrodes and the skin. Electrically speaking, when the fluid is deployed, the electrical impedance between each electrode and the skin is reduced. Mechanically speaking, the fluid may be in the form of a low-viscosity gel. As such, it will not flow too far away from the location it is released. The fluid can be used for both the therapy electrodes 504, 508, and for the sensing electrodes 509.

The fluid may be initially stored in a fluid reservoir, not shown in FIG. 5. Such a fluid reservoir can be coupled to the support structure. In addition, a WMS according to embodiments further includes a fluid deploying mechanism 574. The fluid deploying mechanism 574 can be configured to cause at least some of the fluid to be released from the reservoir, and be deployed near one or both of the patient body locations to which the therapy electrodes 504, 508 are configured to be attached to the patient's body. In some embodiments, the fluid deploying mechanism 574 is activated prior to the electrical discharge responsive to receiving an activation signal AS from a WMS processor 530, which is described more fully later in this document.

In some embodiments, the unit 500 also includes a measurement circuit 520, as one or more of its modules working together with its sensors and/or transducers. The measurement circuit 520 senses one or more electrical physiological signals of the patient from the sensor port 519, if provided. Even if the unit 500 lacks a sensor port, the measurement circuit 520 may optionally obtain physiological signals through the nodes 514, 518 instead, when the therapy electrodes 504, 508 are attached to the patient. In these cases, the input reflects an ECG measurement. The patient parameter can be an ECG, which can be sensed as a voltage difference between electrodes 504, 508. In addition, the patient parameter can be an impedance (IMP. or Z), which can be sensed between the electrodes 504, 508 and/or between the connections of the sensor port 519 considered pairwise as channels. Sensing the impedance can be useful for detecting, among other things, whether these electrodes 504, 508 and/or the sensing electrodes 509 are not making good electrical contact with the patient's body at the time. These patient physiological signals may be sensed when available. The measurement circuit 520 can then render or generate information about them as inputs, data, other signals, etc. As such, the measurement circuit 520 can be configured to render a patient input responsive to a patient parameter sensed by a sensor. In some embodiments, the measurement circuit 520 can be configured to render a patient input, such as values of an ECG signal, responsive to the ECG signal sensed by the ECG sensing electrodes 509. More strictly speaking, the information rendered by the measurement circuit 520 is output from it, but this information can be called an input because it is received as an input by a subsequent stage, device or functionality.

The unit 500 also includes a WMS processor 530. The WMS processor 530 may be implemented in a number of ways. Such ways include, by way of example and not of limitation, digital and/or analog processors such as microprocessors and Digital Signal Processors (DSPs), controllers such as microcontrollers, software running in a machine, programmable circuits such as Field Programmable Gate Arrays (FPGAs), Field-Programmable Analog Arrays (FPAAs), Programmable Logic Devices (PLDs), Application Specific Integrated Circuits (ASICs), any combination of one or more of these, and so on.

The WMS processor 530 may include, or have access to, a non-transitory storage medium, such as a memory 538 that is described more fully later in this document. Such a memory can have a non-volatile component for storage of machine-readable and machine-executable instructions. A set of such instructions can also be called a program. The instructions, which may also be referred to as “software,” generally provide functionality by performing acts, operations and/or methods as may be disclosed herein or understood by one skilled in the art in view of the disclosed embodiments. In some embodiments, and as a matter of convention used herein, instances of the software may be referred to as a “module” and by other similar terms. Generally, a module includes a set of the instructions so as to offer or fulfill a particular functionality. Embodiments of modules and the functionality delivered are not limited by the embodiments described in this document.

The WMS processor 530 can be considered to have a number of modules. One such module can be a detection module 532. The detection module 532 can include a Ventricular Fibrillation (VF) detector. The patient's sensed ECG from measurement circuit 520, which can be available as inputs, data that reflect values, or values of other signals, may be used by the VF detector to determine whether the patient is experiencing VF. Detecting VF is useful, because VF typically results in SCA. The detection module 532 can also include a Ventricular Tachycardia (VT) detector for detecting VT, and so on.

Another such module in WMS processor 530 can be an advice module 534, which generates advice for what to do. The advice can be based on outputs of the detection module 532. There can be many types of advice according to embodiments. In some embodiments, the advice is a shock/no shock determination that WMS processor 530 can make, for example via advice module 534. The shock/no shock determination can be made by executing a stored Shock Advisory Algorithm. A Shock Advisory Algorithm can make a shock/no shock determination from one or more ECG signals that are sensed according to embodiments, and determine whether or not a shock criterion is met. The determination can be made from the patient input, e.g. from a rhythm analysis of the sensed ECG signal or otherwise. For example, there can be shock decisions for VF, VT, etc.

In perfect conditions, a very reliable shock/no shock determination can be made from a segment of the sensed ECG signal of the patient. In practice, however, the ECG signal is often corrupted by electrical noise, which makes it difficult to analyze. Too much noise sometimes causes an incorrect detection of a heart arrhythmia, resulting in a false alarm to the patient. Noisy ECG signals may be handled as described in published US patent application No. US 2019/0030351 A1, and No. US 2019/0030352 A1, and which are incorporated herein by reference.

The WMS processor 530 can include additional modules, such as other module 536, for other functions. In addition, if the internal monitoring device 581 is indeed provided, the WMS processor 530 may receive its inputs, etc.

The unit 500 optionally further includes a memory 538, which can work together with the WMS processor 530. The memory 538 may be implemented in a number of ways. Such ways include, by way of example and not of limitation, volatile memories, Nonvolatile Memories (NVM), Read-Only Memories (ROM), Random Access Memories (RAM), magnetic disk storage media, optical storage media, smart cards, flash memory devices, any combination of these, and so on. The memory 538 is thus a non-transitory storage medium. The memory 538, if provided, can include programs for the WMS processor 530, which the WMS processor 530 may be able to read and execute. More particularly, the programs can include sets of instructions in the form of code, which the WMS processor 530 may be able to execute upon reading. Executing is performed by physical manipulations of physical quantities, and may result in functions, operations, processes, acts, actions and/or methods to be performed, and/or the WMS processor 530 to cause other devices or components or blocks to perform such functions, operations, processes, acts, actions and/or methods. The programs can be operational for the inherent needs of the WMS processor 530, and can also include protocols and ways that decisions can be made by the advice module 534. In addition, the memory 538 can store prompts for the user 582, if this user is a local rescuer. Moreover, the memory 538 can store data. This data can include patient data, system data and environmental data, for example as learned by the internal monitoring device 581 and the outside monitoring device 180. The data can be stored in the memory 538 before it is transmitted out of the unit 500, or be stored there after it is received by the unit 500.

The unit 500 can optionally include a communication module 590, for establishing one or more wired or wireless communication links with other devices of other entities, such as a remote assistance center, Emergency Medical Services (EMS), and so on. The communication links can be used to transfer data and commands. The data may be patient data, event information, therapy attempted, CPR performance, system data, environmental data, and so on. For example, the communication module 590 may transmit wirelessly, e.g. on a daily basis, heart rate, respiratory rate, and other vital signs data to a server accessible over the internet, for instance as described in US 20140043149. This data can be analyzed directly by the patient's physician and can also be analyzed automatically by algorithms designed to detect a developing illness and then notify medical personnel via text, email, phone, etc. The module 590 may also include such interconnected sub-components as may be deemed necessary by a person skilled in the art, for example an antenna, portions of a processor, supporting electronics, outlet for a telephone or a network cable, etc.

The unit 500 may also include a power source 540, which is configured to provide electrical charge in the form of a current. To enable portability of the unit 500, the power source 540 typically includes a battery. Such a battery is typically implemented as a battery pack, which can be rechargeable or not. Sometimes a combination is used of rechargeable and non-rechargeable battery packs. An example of a rechargeable battery 540 was a battery 440 of FIG. 4. Other embodiments of the power source 540 can include an AC power override, for where AC power will be available, an energy-storing capacitor, and so on. Appropriate components may be included to provide for charging or replacing the power source 540. In some embodiments, the power source 540 is controlled and/or monitored by the WMS processor 530.

The unit 500 may additionally include an energy storage module 550. The energy storage module 550 can be coupled to receive the electrical charge provided by the power source 540. The energy storage module 550 can be configured to store the electrical charge received by the power source 540. As such, the energy storage module 550 is where some electrical energy can be stored temporarily in the form of an electrical charge, when preparing it for discharge to administer a shock. In embodiments, the module 550 can be charged from the power source 540 to the desired amount of energy, for instance as controlled by the WMS processor 530. In typical implementations, the module 550 includes a capacitor 552, which can be a single capacitor or a system of capacitors, and so on. In some embodiments, the energy storage module 550 includes a device that exhibits high power density, such as an ultracapacitor. As described above, the capacitor 552 can store the energy in the form of an electrical charge, for delivering to the patient.

As mentioned above, the patient is typically shocked when the shock criterion is met. In particular, in some embodiments the WMS processor 530 is configured to determine from the patient input whether or not a shock criterion is met, and cause, responsive to the shock criterion being met, at least some of the electrical charge stored in the module 550 to be discharged via the therapy electrodes 104, 108 through the ambulatory patient 82 while the support structure is worn by the ambulatory patient 82 so as to deliver the shock 111 to the ambulatory patient 82. Delivering the electrical charge is also known as discharging and shocking the patient.

For causing the discharge, the unit 500 moreover includes a discharge circuit 555. When the decision is to shock, the WMS processor 530 can be configured to control the discharge circuit 555 to discharge through the patient at least some of all of the electrical charge stored in the energy storage module 550, especially in a desired waveform. When the decision is to merely pace, i.e., to deliver pacing pulses, the WMS processor 530 can be configured to control the discharge circuit 555 to discharge through the patient at least some of the electrical charge provided by the power source 540. Since pacing requires lesser charge and/or energy than a defibrillation shock, in some embodiments pacing wiring 541 is provided from the power source 540 to the discharge circuit 555. The pacing wiring 541 is shown as two wires that bypass the energy storage module 550, and only go through a current-supplying circuit 558. As such, the energy for the pacing is provided by the power source 540 either via the pacing wiring 541, or through the energy storage module 550. And, in some embodiments where only a pacer is provided, the energy storage module 550 may not be needed if enough pacing current can be provided from the power source 540. Either way, discharging can be to the nodes 514, 518, and from there to the therapy electrodes 504, 508, so as to cause a shock to be delivered to the patient. The circuit 555 can include one or more switches 557. The switches 557 can be made in a number of ways, such as by an H-bridge, and so on. In some embodiments, different ones of the switches 557 may be used for a discharge where a defibrillation shock is caused to be delivered, than for a discharge where the much weaker pacing pulses are caused to be delivered. The circuit 555 could also be thus controlled via the WMS processor 530, and/or the user interface 580.

The pacing capability can be implemented in a number of ways. ECG sensing may be done in the processor, as mentioned elsewhere in this document, or separately, for demand or synchronous pacing. In some embodiments, however, pacing can be asynchronous. Pacing can be software controlled, e.g., by managing the defibrillation path, or a separate pacing therapy circuit (not shown) could be included, which can receive the ECG sensing, via the circuit 520 or otherwise.

A time waveform of the discharge may be controlled by thus controlling discharge circuit 555. The amount of energy of the discharge can be controlled by how much energy storage module has been charged, and also by how long the discharge circuit 555 is controlled to remain open.

The unit 500 can optionally include other components.

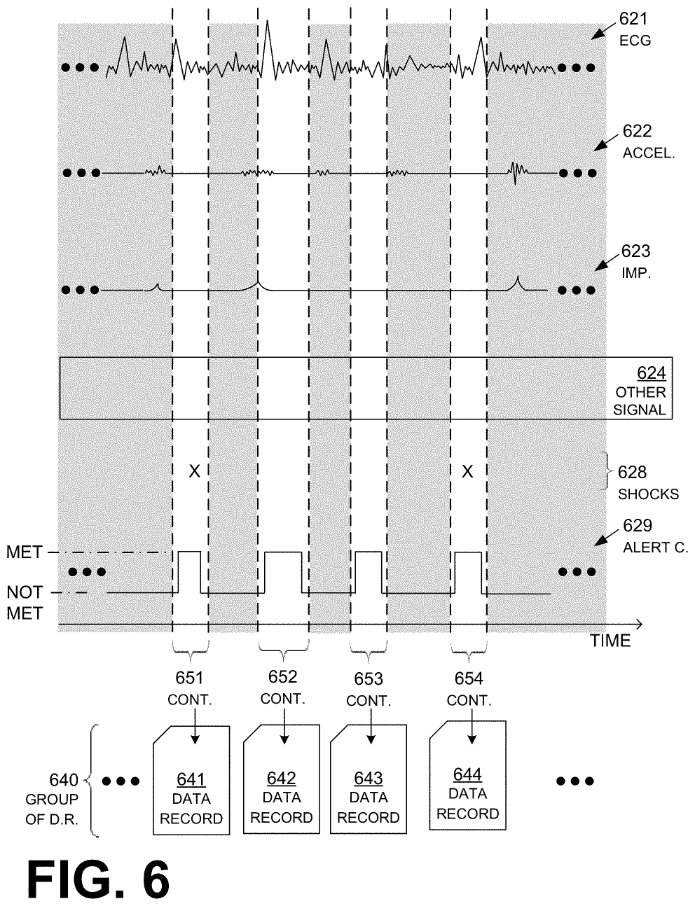

Referring now to FIG. 6, the creation of data records is described. It will be understood that, for the sample embodiments shown, the data records may be created advantageously by the WMS processor 530.

FIG. 6 shows a sample patient input which, in this example, is a waveform 621 of an ECG signal, a waveform 622 of an accelerometer signal, and a waveform 623 of an impedance signal. These waveforms are provided against a time axis that provides a reference for time data. In particular, the WMS processor 530 may further include a clock that provides the time data for these waveforms. While waveforms of signals are shown, their values are meant at the times of the time data. These waveforms are shown beginning and ending with dot-dot-dot, to indicate that they have began before the depicted time portion, and end after the depicted time portion. FIG. 6 further shows optional data from another signal 624, which can be one more signal of the patient or the environment, as deemed suitable. Additional patient input is time instances, shown by “X”s as a group 628 of when shocks were delivered to the patient. It should be understood that the sample values of the waveforms of the example of FIG. 6 are for discussion only, and not intended to be individually realistic depictions of such waveforms for an actual patient. Other versions of the patient input may include fewer, more, and/or different patient parameters than the ones shown in FIG. 6.

FIG. 6 also shows a group 640 of data records 641, 642, 643, 644, . . . . These receive as contents 651, 652, 653, 654 data occurring during vertical time slivers shown on the time axis as not shaded. The remaining times are shown as lightly shaded because they do not become known to the eventual reviewer of the group 640 of data records. The WMS processor 530 can decide when to create these slivers depending on priorly set criteria. In some embodiments, the WMS processor 530 uses an alert criterion when the patient is detected to be having an event or episode of potential interest. For example, the WMS processor 530 can be configured to detect, from the patient input, when an alert criterion is met. This can be accomplished in a number of ways. For instance, when the patient input includes values of the ECG signal 621, the alert criterion can be met responsive to the analyzed ECG signal meeting a tachycardia condition. Such a condition can be ventricular fibrillation (VF), ventricular tachycardia (VT), supraventricular tachycardia (SVT), and so on. Other criteria are described later in this document.

FIG. 6 also shows a sample waveform 629 of a time evolution of when the alert criterion is met and not met. During those times, the WMS processor 530 can be configured to capture at least some of the values of the ECG signal 621, and of additional signals of the patient input. It will be observed that the contents 651, 652, 653, 654 can actually start before the alert criterion is met, by using a memory in a round-robin configuration that writes data, and then over-writes it after some buffering time if the alert criterion has not been met. That buffering time can be two minutes, five minutes, and so on. Similarly, the data collection for the contents 651, 652, 653, 654 can continue for a short time after the alert criterion is no longer met.

The WMS processor 530 can be further configured to create the data records 641, 642, 643, 644 so that the data records 641, 642, 643, 644 are standalone computer files and have as their contents 651, 652, 653, 654 at least some of the values of the waveforms above captured during the non-shaded time slivers. The WMS processor 530 of FIG. 5 can be further configured to cause the data records 641, 642, 643, 644 to be transmitted via the communication module 590. The transmission could be to the pre-analyzing computer system 120, for example as described with reference to FIG. 1 for the data record 140.

Returning to FIG. 1, operations resulting from the instructions 126 being executed, include receiving, by the pre-analyzing computer system 120, the data record 140 that has been caused to be transmitted, for instance via the connection 199. The received data record 140 has contents 150, which have been derived as per the above.

These operations further include parsing the contents 150 of the received data record 140. Parsing can be to identify the individual contents 150; sample such contents were described with reference to FIG. 6, as slivers of waveforms captured during time slivers, and so on.

These operations further include applying a sorting criterion 198 to the parsed contents 150, to determine a given score 160 for the data record 140. The fact that the given score 160 is for the data record 140 is indicated by a dotted arrow. An example is now described.

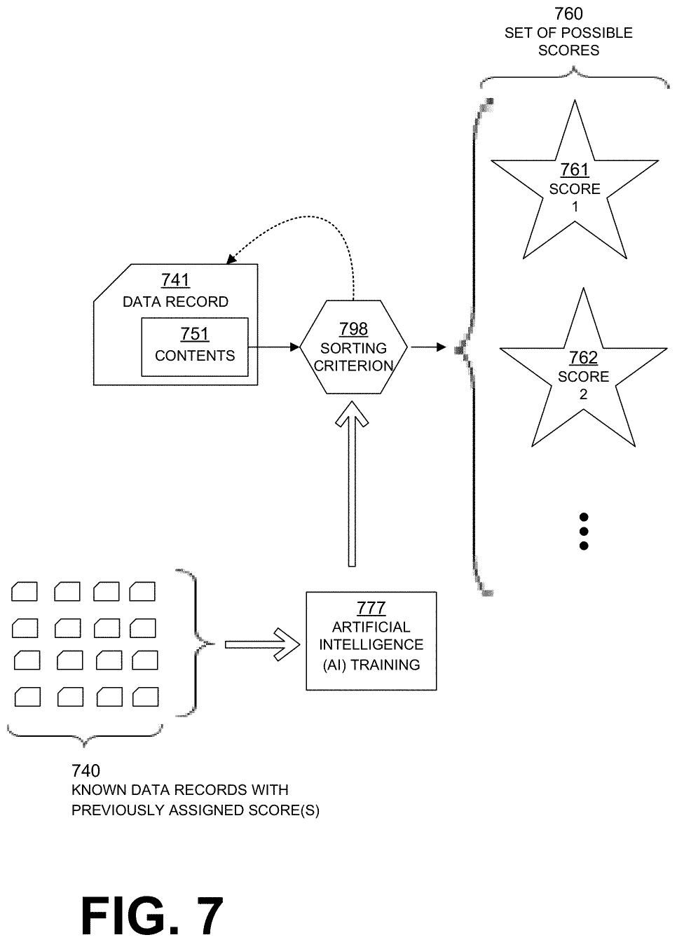

FIG. 7 is a diagram showing a sample data record 741, which has contents 751 that have been parsed. A sorting criterion 798 is applied to the parsed contents 751, which determines a given score for the data record 741. In embodiments, the given score is one of a set 760 of possible scores. The set 760 includes at least a first score 761, a second score 762, and sometimes additional possible scores indicated by dot-dot-dot. As such, in some embodiments the set 760 includes three or more scores. In other embodiments, however, the set 760 includes only and exactly two scores.

The sorting criterion 798 thus generates the score that is determined for, and given to the data record 741. For this reason the sorting criterion 798 can also be thought of as a scoring criterion. Nevertheless, the term “sorting criterion” is preferred for when it is applied to all the data records of a group of batch, as seen later in this document.

In some embodiments, the sorting criterion 798 has been trained by artificial intelligence (AI) training 777. The AI training can be from a group 740 of other data records, to which scores were assigned previously, perhaps by people with medical training.

Returning to FIG. 1, operations resulting from the instructions 126 being executed may further include performing a characterizing action 171 with reference to the data record 140 responsive to the given score 160 being a first possible score, for instance as determined at the diamond 161. In such embodiments, the characterizing action 171 is not performed with reference to the data record 140 responsive to the given score 160 being the second possible score.

In some embodiments, these operations further include performing an alternative action 172 with reference to the data record 140 responsive to the given score 160 being the second score, for instance as determined at the diamond 162. The alternative action 172 can be different from the characterizing action 171. In such embodiments, the alternative action 172 is not performed with reference to the data record 140 responsive to the given score being the first score. Examples of such actions are provided later in this document.

The sorting criterion 198 can be chosen in view of i) what type of data is in the record 140, as already discussed with reference to FIG. 6, ii) what type of use case is selected, and iii) what is the alert criterion, with its waveform 629 of FIG. 6, that generates the data record 140 in the first place. In fact, if it is desired to review certain types of events for a certain patient, certain types of data should be included in the data record, custom alert criteria can be created, and so on with corresponding characterizing actions. Examples are now described of sorting criteria.

In some embodiments, the sorting criterion includes a noise criterion that is applied to the values of the ECG signal, for the amount of electrical noise that may make an interpretation of the ECG signal not reliable. A number of noise criteria can be developed, and for different types of noise. Where various amounts of noise are present, a threshold can be implemented for PASS/FAIL scoring. The threshold can be trained by the AI training 777, after reviewing sample data records with various amounts of noise, whose PASS/FAIL scores have been determined by humans. More detailed examples are now described.

In some embodiments, the noise criterion includes a High-Frequency noise criterion. This can be implemented, for example, by using what is described in published US patent application US20190030351A1, which is hereby incorporated by reference.

In some embodiments, the noise criterion includes a High-Amplitude noise criterion. This can be implemented, for example, by using what is described in published US patent application US20190030352A1, which is hereby incorporated by reference.

In some embodiments, the sensor includes a motion detector, and the patient input includes a motion detection input derived from the motion detector. A sample motion detection input is waveform 622 in FIG. 6. In such embodiments, the contents 651, 652, 653, 654 of the data record 641, 642, 643, 644 can further include motion data derived from the motion detection input, for instance the non-shaded slivers of waveform 622. In such embodiments, the sorting criterion can include a motion criterion that is applied to the motion data. The motion data can indicate if the patient started moving, or started climbing stairs, or changed posture, such as by standing up or bending down. Such events may temporarily increase the heart rate that is detected in all persons, for which reason such episodes might not be of interest to the clinician about the patient 82. In fact, the detected heart rate may be increased because either the true HR increased or the added noise can increase the detected HR. For example bending causes a loose contact between the electrode and skin and adds motion artifacts to ECG signal. In such embodiments, the motion criterion may include that the motion data indicates start of a new type of motion, and so on.

For these reasons, acceleration changes can be very probative for the sorting criterion. In addition to the factors discussed above, acceleration changes might also not generate a change in the heart rate, if they are the byproduct of the patient 82 being in a car that accelerates and decelerates.

In some embodiments, the sensor includes an impedance detector, and the patient input includes impedance values detected from the impedance detector. A sample variation of impedance values is shown in waveform 623 in FIG. 6. In such embodiments, the contents 651, 652, 653, 654 of the data record 641, 642, 643, 644 can further include at least some of the impedance values, for instance the non-shaded slivers of waveform 623. In such embodiments, the sorting criterion can include an impedance criterion that is applied to the at least some of the impedance values. Often times the impedance change in values can indicate the patient's respiration, for example as described in U.S. Pat. No. 11,052,241 B2.

Various aspects of the waveform 623 may be of interest to the sorting criterion. As such, in some embodiments the impedance criterion includes that the at least some of the impedance values are larger than a threshold, which may indicate that the ECG lead no longer makes contact with the patient's skin. In some embodiments, the impedance criterion includes that the at least some of the impedance values start increasing at a rate faster than a threshold, which may indicate that the ECG lead is about to lose contact with the patient's skin.

In some embodiments, the alert criterion is met when a shock is delivered to the patient. It is often very interesting that a shock is applied to the patient and, having the alert criterion being met due to a shock being delivered helps ensure that a data record will indeed be created for later review, just in case it is not created by other factors. In such embodiments, the contents of the data record further include a time instance of when a shock is delivered to the patient. A group 628 of such time instances was seen in FIG. 6. Further in such embodiments, the sorting criterion may include a shock delivery criterion that is applied to the time instance. The characterizing action may be such as to indicate that this data record includes delivery of a shock.

Embodiments can be adapted to different use cases. For instance, the data records may be pre-analyzed for reviewability by the clinician. For such a use case, the set 760 may include exactly two scores, namely PASS and FAIL. The names of “PASS” and “FAIL” can be engineering terms for internal implementation purposes, and need not be revealed to the user in a UI. Instead, more appropriate labels can be developed for the reviewing clinicians, such as “useful to review” v. “not useful to review”, “probative of the state of the patient” v. “not probative”, and so on.

In embodiments where the pre-analyzing is for the reviewability of the data records, the FAIL score may be given to data records whose contents are likely not interpretable reliably due to noise or likely of low interest. As such, the scoring criterion can be set to recognize their contents and give them a FAIL score, while giving a PASS score to all other data records. This way, a reviewer will not waste time opening and trying to review data records that will benefit them very little.

Embodiments can be adapted for other uses. For instance, they can be adapted by type of event that merited the alert criterion being met. Or, they can differentiate by type of why a FAIL score is given, e.g. different for ECG noise than for those of likely of low interest. Also different for different types of ECG noise, and so on.

The characterizing action can be adapted for how data records are to be reviewed by the clinician. There are various possibilities, and examples are now described.

In some embodiments, the characterizing action includes placing a data record in a bucket together with another data record that has the same given score. And, the alternative action is to place data records with a different score in a different bucket. When this is pre-analyzing is done to all the data records in a batch or group, sorting results. These buckets are intended first metaphorically, and are implemented in a number of ways, such as similar filenames, same folders, and so on. An example is now described.

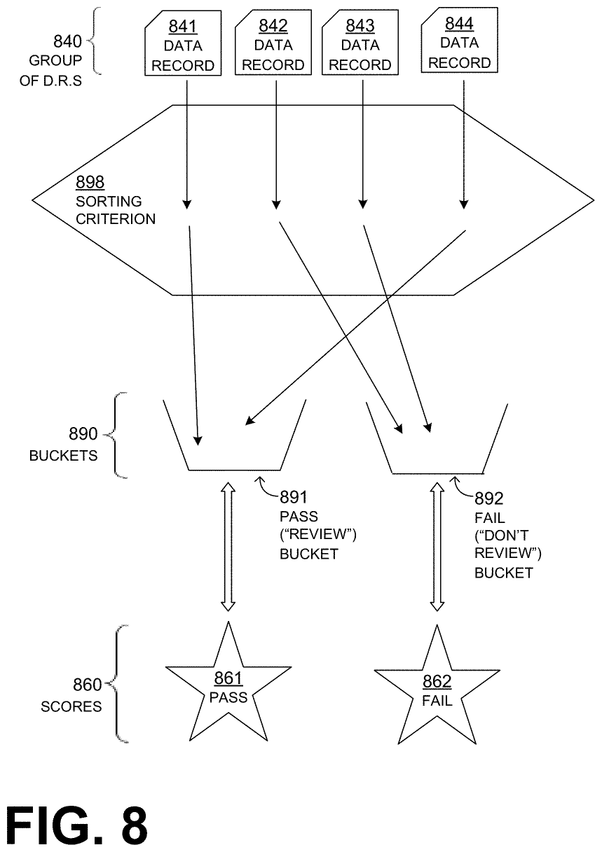

FIG. 8 is a diagram showing a sorting criterion 898 can result in a score of PASS 861 or FAIL 862. These are the only possible scores of a set 860.

A set 890 of buckets or categories is defined. The set 890 includes a PASS (“Review”) bucket 891, and a FAIL (“Don't Review”) bucket 892.

The sorting criterion 898 is applied to the contents (not shown separately) of data records 841, 842, 843, 844. Of those, the data records 841, 844 receive a score of PASS and are placed in the PASS bucket 891, which can also be an instance of the characterizing action. The data records 842, 843 receive a score of FAIL and are placed in the FAIL bucket 892, which can also be an instance of the alternative action.

In some embodiments, the characterizing action includes creating a scores record indicative of the given score. There are embodiments for various types of scores records, such as entering score entries into a standalone scores computer file, entering notations in the filenames of the data record, entering scoring notes in the contents of the data records, and scores records created by virtue of which folders the data records are placed and how. Also, combinations of the above are possible. Examples are now described.

In some embodiments, the pre-analyzing computer system 120 stores a standalone scores computer file, which is not shown in FIG. 1. Storing can be in a memory, such as the storage medium 124. The standalone scores computer file can be distinct from the data record 140. In such embodiments, the characterizing action 171 may include making a score entry indicative of the given score 160 into the scores computer file. Examples are now described.

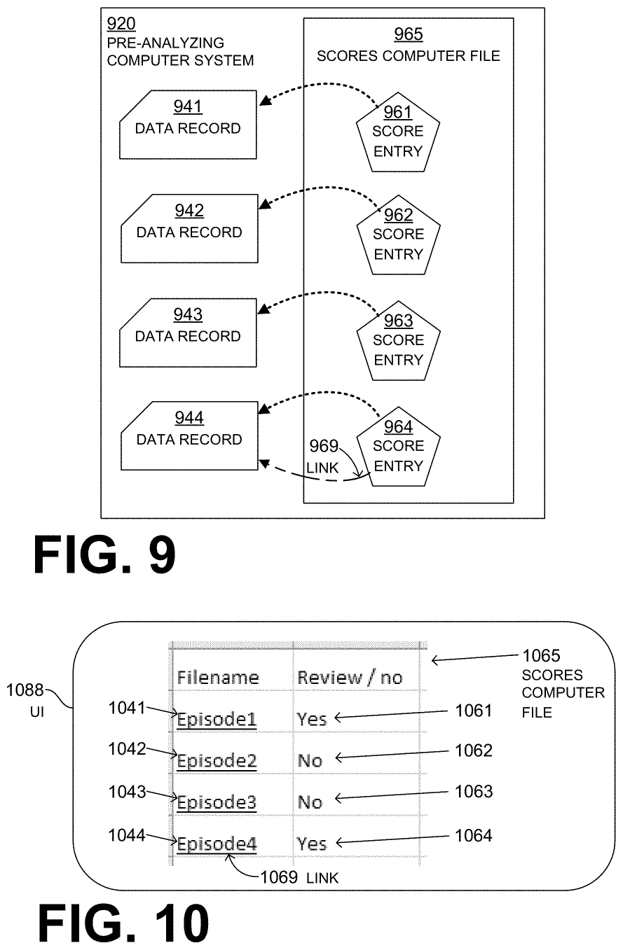

FIG. 9 is a diagram showing a pre-analyzing computer system 920, which can be as the pre-analyzing computer system 120. The pre-analyzing computer system 920 has received four sample data records 941, 942, 943, 944. As mentioned above, there may be many more data records than just four.

A sample scores computer file 965 is distinct, different from the data records 941, 942, 943, 944. The received data records 941, 942, 943, 944 have been pre-analyzed, and for which respective scores (not shown) have been determined and given. Score entries 961, 962, 963, 964 are made into the scores computer file 965. The score entries 961, 962, 963, 964 are indicative of the given scores. The score entries 961, 962, 963, 964 are thus for the data records 941, 942, 943, 944, as indicated by the four dotted arrows. In some embodiments, the score entry 964 includes a hyperlink 969 that points to the data record 944. Of course, such hyperlinks can be provided for all the score entries.

FIG. 10 shows a UI 1088, which can be as UI 188. The UI 1088 shows a portion of a sample scores computer file 1065. The scores computer file 1065 can be as described for the scores computer file 965. The scores computer file 1065 has a table with a left column that lists filenames 1041, 1042, 1043, 1044 of respective data records. In this embodiment, all the filenames include hyperlinks that point to the respective data records. Of those, only link 1069 is indicated with a reference numeral.

The table also has a right column that lists respective score entries 1061, 1062, 1063, 1064 for the filenames of the left column. The score entries 1061, 1062, 1063, 1064 have values “Yes” or “No”, which are indicative of the respective scores (“PASS”/“FAIL”).

In some embodiments, the received data record has a first filename, and the characterizing action includes changing the first filename into a second filename that is different from the first filename. In such embodiments, the second filename sometimes indicates the given score. In such embodiments, caution should be undertaken with the filename change, to not disrupt any linking such as was described for FIG. 9 and FIG. 10. Examples are now described.

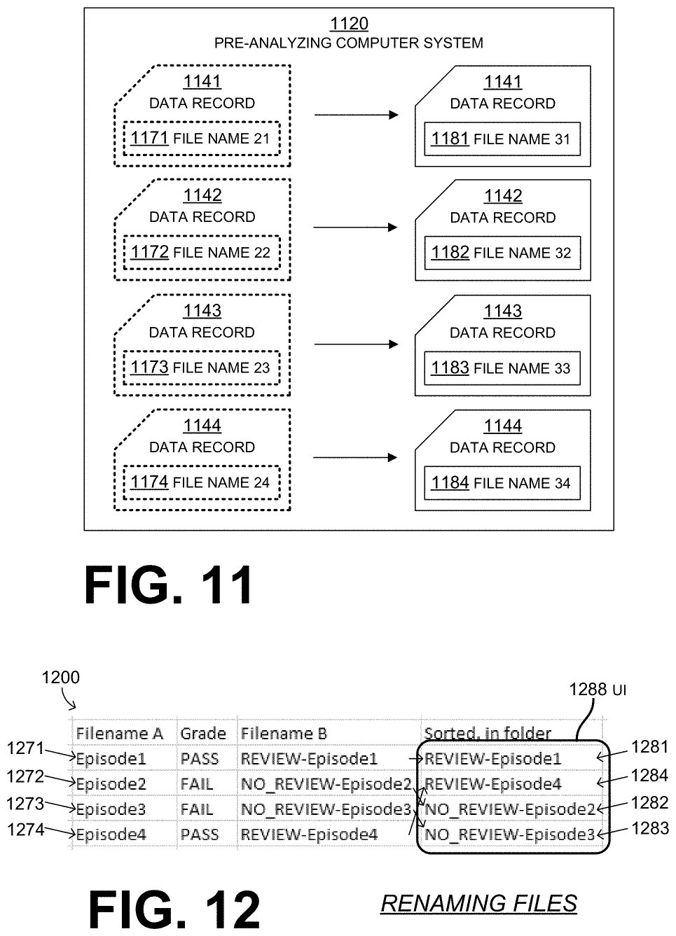

FIG. 11 is a diagram showing a pre-analyzing computer system 1120, which can be as the pre-analyzing computer system 120. The pre-analyzing computer system 1120 has received four sample data records 1141, 1142, 1143, 1144.

The data record 1141 has a first filename 21 1171, which is changed to a second filename 31 1181. The second filename 31 is different from the first filename 21. The data record 1142 has a first filename 22 1172, which is changed to a second filename 32 1182. The data record 1143 has a first filename 23 1173, which is changed to a second filename 33 1183. The data record 1144 has a first filename 24 1174, which is changed to a second filename 34 1184.

FIG. 12 shows a table 1200. The table 1200 has a first column with first filenames A 1271, 1272, 1273, 1274 of four sample data records. The table 1200 also has a second column with sample scores given to the data records of the first column. The table 1200 also has a third column showing how the filenames of the first column have changed to second filenames B. Changing the file name of a data record was first introduced with reference to FIG. 11. It will be appreciated that, in the example of FIG. 12, the second filenames indicate the given score, namely “REVIEW” for “PASS” and “NO REVIEW” for “FAIL”.

The table 1200 also has a fourth column, where the second filenames of the third column are repeated as 1281, 1284, 1282, 1283, except that they have been sorted in reverse alphabetical but not numeric order. The last column is also what might appear to a clinician in a UI 1288, which can be as described for UI 188. While FIG. 12 was an example with only four files, there could be many tens of files, even hundreds of files, and the different filenames may make it easier for the reviewer to identify quickly those of interest, especially if sorted as shown in FIG. 12.

In some embodiments, the characterizing action includes adding to the contents of the received data record at least one scoring note. In such embodiments, the scoring note can be indicative of at least one of the given score, of a date in which the given score was determined, and of the pre-analyzing computer system that determined the given score. An example is now described.

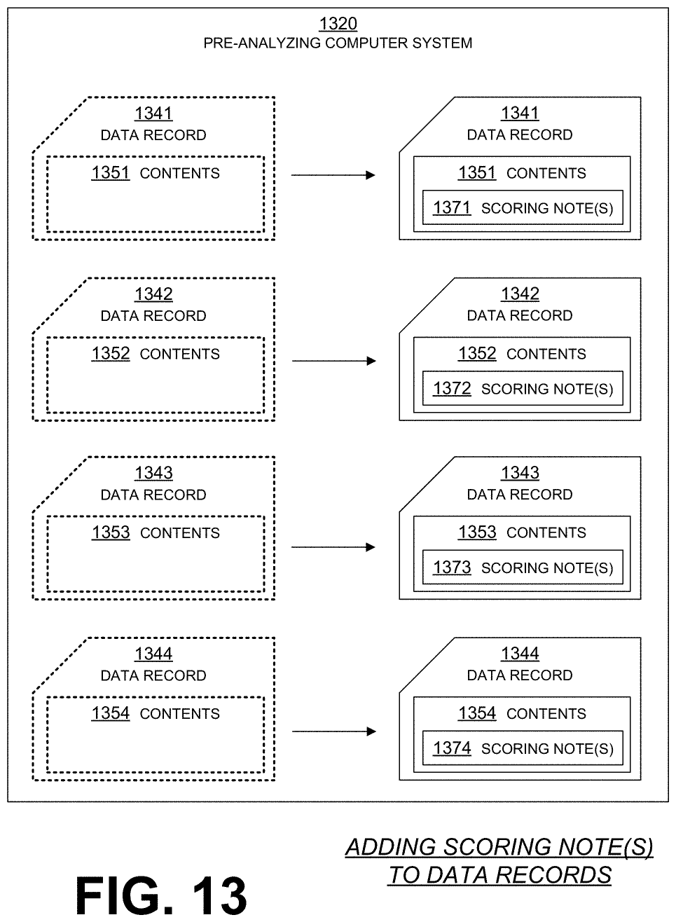

FIG. 13 is a diagram showing a pre-analyzing computer system 1320, which can be as the pre-analyzing computer system 120. The pre-analyzing computer system 1320 has received four sample data records 1341, 1342, 1343, 1344, which have respective contents 1351, 1352, 1353, 1354. One or more scoring notes 1371 have been added to the contents 1351 of the data record 1341. One or more scoring notes 1372 have been added to the contents 1352 of the data record 1342. One or more scoring notes 1373 have been added to the contents 1353 of the data record 1343. One or more scoring notes 1374 have been added to the contents 1354 of the data record 1344.

It will be appreciated that the scoring notes can be used by the clinician to evaluate the pre-analysis. In some embodiments, even more data can be added to the scoring notes. An example is now described.

In some embodiments, the set 760 includes only two possible scores, such as a first score and a second score. In such embodiments, the operations from executing the instructions 126 may further include: generating, responsive to the given score being determined to be the second score, a score determination record that explains how the determination was made. Moreover, the characterizing action may include, responsive to the given score being determined to be the second score, adding to the contents of the received data record the score determination record. Of course, the score determination record can be added also if the given score is the first score, and so on. An example is now described.

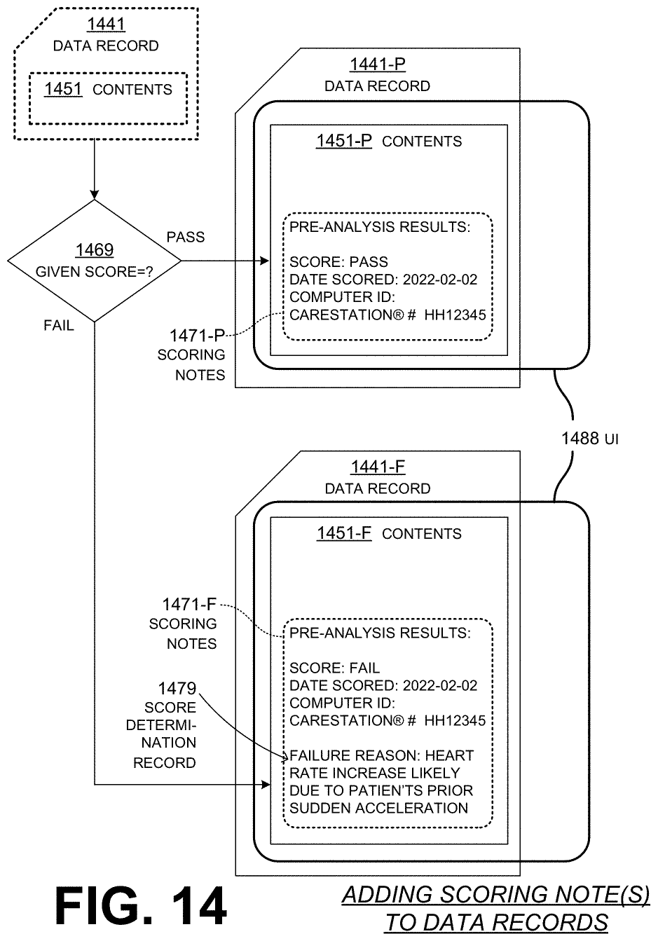

FIG. 14 shows a data record 1441 having contents 1451. At a diamond 1469 it is inquired what was the given score. In this example, only two scores are possible, namely a PASS score (first score) and a FAIL score (second score).

If at the diamond 1469 the given score is PASS, the data record 1441 becomes a data record 1441-P, with new contents 1451-P. Scoring notes 1471-P have been added to the contents 1451-P, which can be visible to a clinician in a first view of a UI 1488 that can be as described for the UI 188.

In this example, these scoring notes 1471-P show the pre-analysis results, with the following elements: the given score (PASS), the date in which the given score was determined (2022-02-02), and the pre-analyzing computer system that determined the given score (CARESTATION® # HH12345).

Alternately, if at the diamond 1469 the given score is FAIL, the data record 1441 becomes a data record 1441-F, with new contents 1451-F. Scoring notes 1471-F have been added to the contents 1451-F, which can be visible to a clinician in an alternate view of the UI 1488.

In this example, these scoring notes 1471-F include the same elements as the scoring notes 1471-P described above, plus they include a score determination record 1479, which explains how the determination was made. From the text of the score determination record 1479, it is apparent that this data record 1441 was legitimately created by the patient's suddenly increased heart rate, but it is of low interest after all because of the explanation given.

In some embodiments, the received data record is stored in a records folder. This records folder may be the folder into which the data record was received in, or another folder. In such embodiments, the characterizing action includes moving the received data record from the records folder into a first folder that is different from the records folder. Moreover, in some of these embodiments, the operations from executing the instructions 126 may further include moving, responsive to the given score being the second score, the received data record into a second folder that is different from the records folder and from the first subfolder. An example is now described.

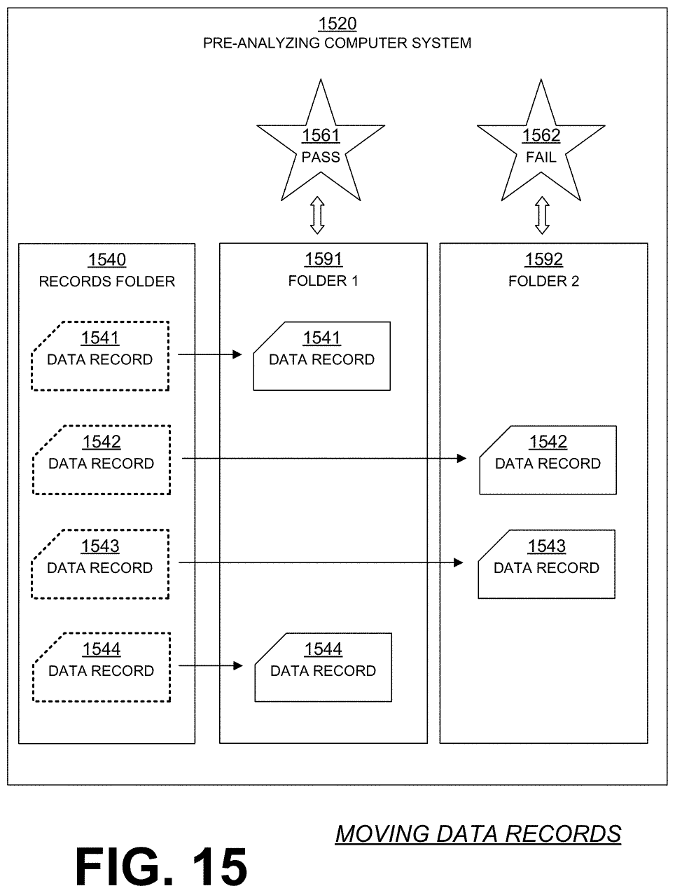

FIG. 15 is a diagram showing a pre-analyzing computer system 1520, which can be as the pre-analyzing computer system 120. The pre-analyzing computer system 1520 has received four sample data records 1541, 1542, 1543, 1544, which it stores in a records folder 1540 before the pre-analyzing.

In this example, only two scores are possible, namely a PASS score 1561 (first score) and a FAIL score 1562 (second score). A first folder 1 1591 is associated with the PASS score 1561, and a second folder 2 1592 is associated with the FAIL score 1562. The first folder 1 1591 and the second folder 2 1592 are different from the records folder 1540, although they can all be in the same set of common folders and/or subfolders.

In this example, the characterizing action includes moving the data records 1541, 1544 from the records folder 1540 into the first folder 1 1591. These are the data records that received PASS scores, and often these are the only ones that the clinician is interested in reviewing.

Further in this example, the operations from executing the instructions 126 included moving the data records 1542, 1543 from the records folder 1540 into the second folder 2 1592. These are the data records that received FAIL scores, and often the clinician is not interested in reviewing them. Moving the data records 1542, 1543 into the second folder 2 1592 was an example of the alternative action 172.

In some embodiments, the received data record is stored in a records folder. This records folder may be the folder into which the data record was received in, or another folder. In such embodiments, the characterizing action may include preparing a standalone episode review file distinct from the received data record, the episode review file including at least some of the ECG values of the received data record. In such embodiments, the characterizing action may further include storing the episode review file in a review folder that is different from the records folder. An example is now described.

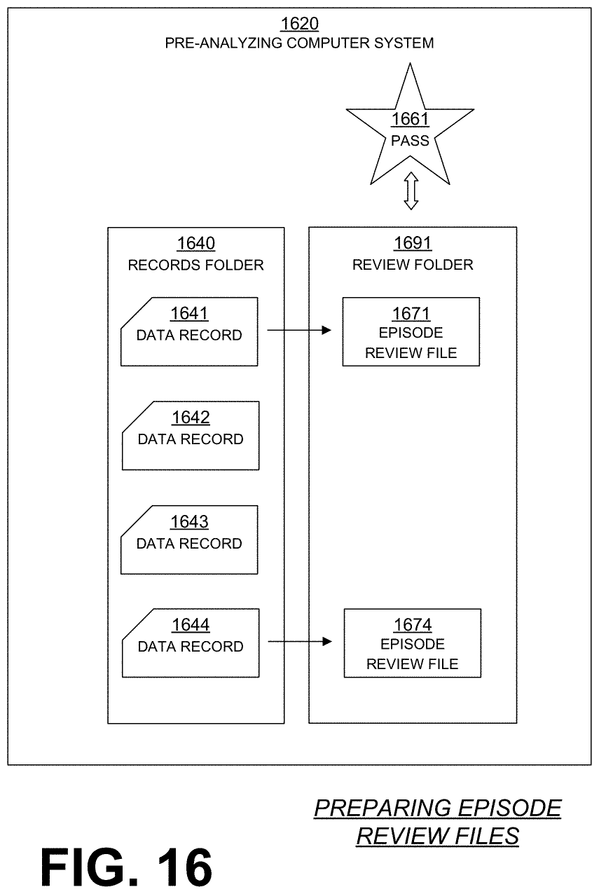

FIG. 16 is a diagram showing a pre-analyzing computer system 1620, which can be as the pre-analyzing computer system 120. The pre-analyzing computer system 1620 has received four sample data records 1641, 1642, 1643, 1644, which it stores in a records folder 1640 before the pre-analyzing.

In this example, only two scores are possible, namely a PASS score 1661 (first score) and a FAIL score (not shown). A review folder 1691 is associated with the PASS score 1661. The review folder 1691 is different from the records folder 1640, although they can both be in the same set of common folders and/or subfolders.

In this example, the characterizing action included preparing standalone episode review files 1671, 1674, which are distinct from the received data records 1641, 1644. Preparing can be by copying, in whole or in part, adding notes, and so on. The characterizing action included storing the episode review files 1671, 1674 in the review folder 1691. This example can be for reviewing all episodes that passed, or all episodes with a certain attribute, which have been thus sorted by appropriately choosing the sorting criterion 198.

The devices and/or systems mentioned in this document may perform functions, processes, acts, operations, actions and/or methods. These functions, processes, acts, operations, actions and/or methods may be implemented by one or more devices that include logic circuitry. A single such device can be alternately called a computer, and so on. It may be a standalone device or computer, such as a general-purpose computer, or part of a device that has and/or can perform one or more additional functions. The logic circuitry may include a processor and non-transitory computer-readable storage media, such as memories, of the type described elsewhere in this document. Often, for the sake of convenience only, it is preferred to implement and describe a program as various interconnected distinct software modules or features. These, along with data are individually and also collectively known as software. In some instances, software is combined with hardware, in a mix called firmware.

Moreover, methods and algorithms are described below. These methods and algorithms are not necessarily inherently associated with any particular logic device or other apparatus. Rather, they are advantageously implemented by programs for use by a computing machine, such as a general-purpose computer, a special purpose computer, a microprocessor, a processor such as described elsewhere in this document, and so on.

This detailed description may include flowcharts, display images, algorithms, and symbolic representations of program operations within at least one computer readable medium. An economy may be achieved in that a single set of flowcharts can be used to describe both programs, and also methods. So, while flowcharts describe methods in terms of boxes, they may also concurrently describe programs.

Methods are now described. It will be recognized that a number of these methods may be the previously described operations resulting from the instructions 126 being executed.

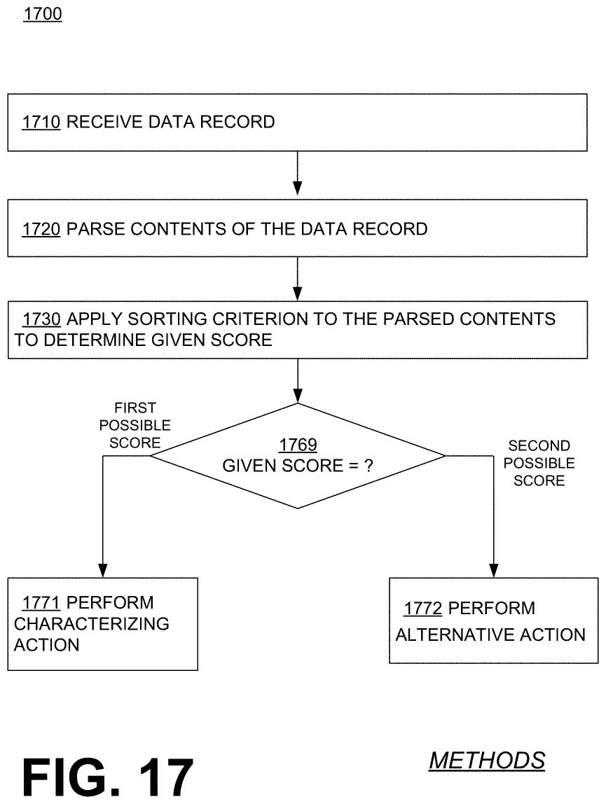

FIG. 17 shows a flowchart 1700 for describing methods according to embodiments.

According to a first operation 1710, a data record may be received, which has been caused to be transmitted.

According to another operation 1720, contents of the received data record may be parsed. The contents may be as shown in FIG. 6.

According to another operation 1730, a sorting criterion may be applied to the parsed contents, to determine a given score for the data record. The given score can be one of a set of possible scores that includes at least a first score and a second score.

According to another operation 1769, it can be inquired what the given score is. The choice may be between a first possible score, a second possible score, and so on.

If at the operation 1769 it is determined that the given score is the first possible score then, according to another operation 1771, a characterizing action may be performed with reference to the data record. In other words, the characterizing action may be performed responsive to the given score being the first score. In some embodiments, the characterizing action is not performed if the given score is not the first possible score, for instance if it is the second possible score.

If at the operation 1769 it is determined that the given score is the second possible score then, according to another operation 1772, an alternative action may be performed with reference to the data record. In other words, the alternative action may be performed responsive to the given score being the second score. In some embodiments, the alternative action is not performed if the given score is not the second possible score, for instance if it is the first possible score. The alternative action is different than the characterizing action, either in type of action or in the form of records created.

In the methods described above, each operation can be performed as an affirmative act or operation of doing, or causing to happen, what is written that can take place. Such doing or causing to happen can be by the whole system or device, or just one or more components of it. It will be recognized that the methods and the operations may be implemented in a number of ways, including using systems, devices and implementations described above. In addition, the order of operations is not constrained to what is shown, and different orders may be possible according to different embodiments. Examples of such alternate orderings may include overlapping, interleaved, interrupted, reordered, incremental, preparatory, supplemental, simultaneous, reverse, or other variant orderings, unless context dictates otherwise. Moreover, in certain embodiments, new operations may be added, or individual operations may be modified or deleted. The added operations can be, for example, from what is mentioned while primarily describing a different system, apparatus, device or method.

At least one of the methods of this description, when implemented by a computer, can be performed differently at the rate of at least 10 times per second.

A person skilled in the art will be able to practice the present invention in view of this description, which is to be taken as a whole. Details have been included to provide a thorough understanding. In other instances, well-known aspects have not been described, in order to not obscure unnecessarily this description.

Some technologies or techniques described in this document may be known. Even then, however, it does not necessarily follow that it is known to apply such technologies or techniques as described in this document, or for the purposes described in this document.

This description includes one or more examples, but this fact does not limit how the invention may be practiced. Indeed, examples, instances, versions or embodiments of the invention may be practiced according to what is described, or yet differently, and also in conjunction with other present or future technologies. Other such embodiments include combinations and sub-combinations of features described herein, including for example, embodiments that are equivalent to the following: providing or applying a feature in a different order than in a described embodiment; extracting an individual feature from one embodiment and inserting such feature into another embodiment; removing one or more features from an embodiment; or both removing a feature from an embodiment and adding a feature extracted from another embodiment, while providing the features incorporated in such combinations and sub-combinations.

In general, the present disclosure reflects preferred embodiments of the invention. The attentive reader will note, however, that some aspects of the disclosed embodiments extend beyond the scope of the claims. To the respect that the disclosed embodiments indeed extend beyond the scope of the claims, the disclosed embodiments are to be considered supplementary background information and do not constitute definitions of the claimed invention.

In this document, the phrases “constructed to”, “adapted to” and/or “configured to” denote one or more actual states of construction, adaptation and/or configuration that is fundamentally tied to physical characteristics of the element or feature preceding these phrases and, as such, reach well beyond merely describing an intended use. Any such elements or features can be implemented in a number of ways, as will be apparent to a person skilled in the art after reviewing the present disclosure, beyond any examples shown in this document.

Incorporation by reference: References and citations to other documents, such as patents, patent applications, patent publications, journals, books, papers, web contents, have been made throughout this disclosure. All such documents are hereby incorporated herein by reference in their entirety for all purposes.

Parent patent applications: Any and all parent, grandparent, great-grandparent, etc. patent applications, whether mentioned in this document or in an Application Data Sheet (“ADS”) of this patent application, are hereby incorporated by reference herein as originally disclosed, including any priority claims made in those applications and any material incorporated by reference, to the extent such subject matter is not inconsistent herewith.

Reference numerals: In this description a single reference numeral may be used consistently to denote a single item, aspect, component, or process. Moreover, a further effort may have been made in the preparation of this description to use similar though not identical reference numerals to denote other versions or embodiments of an item, aspect, component or process that are identical or at least similar or related. Where made, such a further effort was not required, but was nevertheless made gratuitously so as to accelerate comprehension by the reader. Even where made in this document, such a further effort might not have been made completely consistently for all of the versions or embodiments that are made possible by this description. Accordingly, the description controls in defining an item, aspect, component or process, rather than its reference numeral. Any similarity in reference numerals may be used to infer a similarity in the text, but not to confuse aspects where the text or other context indicates otherwise.

The claims of this document define certain combinations and subcombinations of elements, features and acts or operations, which are regarded as novel and non-obvious. The claims also include elements, features and acts or operations that are equivalent to what is explicitly mentioned. Additional claims for other such combinations and subcombinations may be presented in this or a related document.

These claims are intended to encompass within their scope all changes and modifications that are within the true spirit and scope of the subject matter described herein. The terms used herein, including in the claims, are generally intended as “open” terms. For example, the term “including” should be interpreted as “including but not limited to,” the term “having” should be interpreted as “having at least,” etc. If a specific number is ascribed to a claim recitation, this number is a minimum but not a maximum unless stated otherwise. For example, where a claim recites “a” component or “an” item, it means that the claim can have one or more of this component or this item.

In construing the claims of this document, the inventor(s) invoke 35 U.S.C. § 112(f) only when the words “means for” or “steps for” are expressly used in the claims. Accordingly, if these words are not used in a claim, then that claim is not intended to be construed by the inventor(s) in accordance with 35 U.S.C. § 112(f).

1. A pre-analyzing computer system for pre-analyzing and characterizing a data record of a parameter of an ambulatory patient, the data record created by a wearable medical system (WMS) worn by the patient, the WMS including at least: an energy storage module configured to store an electrical charge, an electrode, a support structure configured to be worn by the ambulatory patient so as to maintain the electrode on a body of the ambulatory patient, a sensor configured to sense a parameter of the patient, the parameter including an Electrocardiogram (ECG) signal of the patient, a measurement circuit configured to render a patient input responsive to the sensed parameter, the patient input including values for the ECG signal, and a WMS processor configured to: determine from the patient input whether or not a shock criterion is met, and cause, responsive to the shock criterion being met, at least some of the stored electrical charge to be discharged via the therapy electrode through the ambulatory patient while the support structure is worn by the ambulatory patient so as to deliver a shock to the ambulatory patient, detect, from the patient input, when an alert criterion is met, capture at least some of the values of the ECG signal when the alert criterion is met, create the data record so that the data record is a standalone computer file and has as contents at least some of the captured values, and cause the data record to be transmitted to the pre-analyzing computer system, the pre-analyzing computer system including at least: one or more pre-analyzing computer system processors distinct from the WMS processor and not controlled by the WMS processor; and a non-transitory computer-readable pre-analyzing storage medium having stored thereon instructions which, when executed by the one or more computer pre-analyzing system processors, result in operations including at least:
receiving, by the pre-analyzing computer system, the data record that has been caused to be transmitted;
parsing the contents of the received data record;
applying a sorting criterion to the parsed contents to determine a given score for the data record, the given score being one of a set including at least a first score and a second score; and
performing a characterizing action with reference to the data record responsive to the given score being the first score, and not performing the characterizing action with reference to the data record responsive to the given score being the second score.

2. The pre-analyzing computer system of claim 1, in which:
the set includes exactly two scores.

3. The pre-analyzing computer system of claim 1, in which:
the set includes three or more scores.

4. The pre-analyzing computer system of claim 1, in which:
the sorting criterion has been trained by artificial intelligence training from other data records to which scores were assigned previously.

5. The pre-analyzing computer system of claim 1, in which:
the operations further include:
performing an alternative action with reference to the data record responsive to the given score being the second score, and not performing the alternative action with reference to the data record responsive to the given score being the first score, the alternative action being different from the characterizing action.

6. The pre-analyzing computer system of claim 1, in which:
the sorting criterion includes a noise criterion that is applied to the values of the ECG signal.

7. The pre-analyzing computer system of claim 6, in which:
the noise criterion includes a High-Frequency noise criterion.

51. A method for a pre-analyzing computer system to pre-analyze and characterize a data record of a parameter of an ambulatory patient, the data record created by a wearable medical system (WMS) worn by the patient, the WMS including at least: an energy storage module configured to store an electrical charge, an electrode, a support structure configured to be worn by the ambulatory patient so as to maintain the electrode on a body of the ambulatory patient, a sensor configured to sense a parameter of the patient, the parameter including an Electrocardiogram (ECG) signal of the patient, a measurement circuit configured to render a patient input responsive to the sensed parameter, the patient input including values for the ECG signal, and a WMS processor configured to: determine from the patient input whether or not a shock criterion is met, and cause, responsive to the shock criterion being met, at least some of the stored electrical charge to be discharged via the therapy electrode through the ambulatory patient while the support structure is worn by the ambulatory patient so as to deliver a shock to the ambulatory patient, detect, from the patient input, when an alert criterion is met, capture at least some of the values of the ECG signal when the alert criterion is met, create the data record so that the data record is a standalone computer file and has as contents at least some of the captured values, and cause the data record to be transmitted to the pre-analyzing computer system, the pre-analyzing computer system including at least: one or more pre-analyzing computer system processors distinct from the WMS processor and not controlled by the WMS processor; and a non-transitory computer-readable pre-analyzing storage medium having stored thereon instructions which can be executed by the one or more computer pre-analyzing system processors, the method including at least:
receiving, by the pre-analyzing computer system, the data record that has been caused to be transmitted;
parsing, by the pre-analyzing computer system, the contents of the received data record;
applying, by the pre-analyzing computer system, a sorting criterion to the parsed contents to determine a given score for the data record, the given score being one of a set including at least a first score and a second score; and
performing, by the pre-analyzing computer system, a characterizing action with reference to the data record responsive to the given score being the first score, and not performing the characterizing action with reference to the data record responsive to the given score being the second score.

52. The method of claim 51, in which:
the set includes exactly two scores.

53. The method of claim 51, in which:
the set includes three or more scores.

54. The method of claim 51, in which:
the sorting criterion has been trained by artificial intelligence training from other data records to which scores were assigned previously.

55. The method of claim 51, further including:
performing an alternative action with reference to the data record responsive to the given score being the second score, and not performing the alternative action with reference to the data record responsive to the given score being the first score, the alternative action being different from the characterizing action.

56. The method of claim 51, in which:
the sorting criterion includes a noise criterion that is applied to the values of the ECG signal.

57. The method of claim 56, in which:
the noise criterion includes a High-Frequency noise criterion.

76. A pre-analyzing computer system for pre-analyzing and characterizing a data record of a parameter of an ambulatory patient, the data record created by a wearable medical system (WMS) worn by the patient, the WMS including at least: a support structure configured to be worn by the ambulatory patient so as to maintain a sensor on a body of the ambulatory patient, the sensor configured to sense a parameter of the patient, the parameter including an Electrocardiogram (ECG) signal of the patient, a measurement circuit configured to render a patient input responsive to the sensed parameter, the patient input including values for the ECG signal, and a WMS processor configured to: determine from the patient input whether or not an arrhythmia criterion is met, and cause, responsive to the arrhythmia criterion being met, at least some of the values of the ECG signal to be captured, create the data record so that the data record is a standalone computer file and has as contents at least some of the captured values, and cause the data record to be transmitted to the pre-analyzing computer system, the pre-analyzing computer system including at least: one or more pre-analyzing computer system processors distinct from the WMS processor and not controlled by the WMS processor; and a non-transitory computer-readable pre-analyzing storage medium having stored thereon instructions which, when executed by the one or more computer pre-analyzing system processors, result in operations including at least:
receiving, by the pre-analyzing computer system, the data record that has been caused to be transmitted;
parsing the contents of the received data record;
applying a sorting criterion to the parsed contents to determine a given score for the data record, the given score being one of a set including at least a first score and a second score; and
performing a characterizing action with reference to the data record responsive to the given score being the first score, and not performing the characterizing action with reference to the data record responsive to the given score being the second score.

77. The pre-analyzing computer system of claim 76, in which:
the set includes exactly two scores.

78. The pre-analyzing computer system of claim 76, in which:
the set includes three or more scores.

79. The pre-analyzing computer system of claim 76, in which:
the sorting criterion has been trained by artificial intelligence training from other data records to which scores were assigned previously.

80. The pre-analyzing computer system of claim 76, in which:
the operations further include:
performing an alternative action with reference to the data record responsive to the given score being the second score, and not performing the alternative action with reference to the data record responsive to the given score being the first score, the alternative action being different from the characterizing action.

81. The pre-analyzing computer system of claim 76, in which:
the sorting criterion includes a noise criterion that is applied to the values of the ECG signal.

82. The pre-analyzing computer system of claim 81, in which:
the noise criterion includes a High-Frequency noise criterion.
